(19) United States
(12) Patent Application Publication
(10) Pub. No.: US 2026/0286390 A1
(43) Pub. Date: Sep. 24, 2026

(54) GENE THERAPY FOR TREATING LIMB GIRDLE MUSCULAR DYSTROPHY R9 AND CONGENITAL MUSCULAR DYSTROPHY 1C

(71) Applicant: Research Institute at Nationwide Children's Hospital, Inc., Columbus, OH (US)

(72) Inventor: Paul Martin, Columbus, OH (US)

(21) Appl. No.: 19/476,574

(22) PCT Filed: Apr. 17, 2024
(86) PCT No.: PCT/US24/25062

§ 371 Date: Oct. 17, 2025

Related U.S. Application Data
(60) Provisional application No. 63/460,124, filed on Apr. 18, 2023.

(51) Int. Cl. C12N 15/86; A61K 38/17; A61K 48/00; A61P 21/00; C07K 14/47

(52) U.S. Cl. CPC C12N 15/86; A61K 38/1709; A61K 48/0033; A61K 48/0058; A61P 21/00; C07K 14/47; C12N 2750/14143; C12N 2830/008; C12N 2830/50

(57) ABSTRACT

The invention described herein provides gene therapy vectors, such as adeno-associated virus (AAV) vectors, that co-express two or more GDIs. The vectors of the invention can be broadly used to treat a number of genetic disorders such as trinucleotide repeat expansion disorders.

This application claims priority to U.S. Provisional Patent Application No. 63/460,124, filed Apr. 18, 2023, which is incorporated herein by reference in its entirety.

### INCORPORATION BY REFERENCE OF THE SEQUENCE LISTING

This application contains, as a separate part of the disclosure, a Sequence Listing in computer-readable form which is incorporated by reference in its entirety and identified as follows: 59037_SeqListing.xml; Size: 26,083 bytes; Created: Apr. 17, 2024.

### FIELD OF THE INVENTION

The present invention relates to methods and materials for treating Limb Girdle Muscular Dystrophy R9 (LGMDR9) and Congenital Muscular Dystrophy 1C (MDC1C) using a bicistronic recombinant adeno-associated virus encoding fukutin-related protein (FKRP) and follistatin (FST).

### BACKGROUND

Gene replacement therapy utilizing Adeno Associated Virus (AAV) has now been shown to significantly improve patient outcomes in several genetic disorders, including hemophilia A, Leber's congenital amaurosis, Duchenne muscular dystrophy, and Spinal Muscular Atrophy type 1. In all of these examples, a normal, or miniaturized normal, copy of the mutated gene that causes the disease has been introduced to replace lost gene function. While such an approach can have a profound impact on disease progression, gene replacement typically does not reverse disease that is already present. Such shortcomings can be very significant in degenerative diseases such muscular dystrophy, where muscle weakness is most often caused by the progressive loss of muscle tissue. While in theory gene replacement could entirely prevent muscular dystrophy if given at a stage prior to disease onset, most gene therapies, particularly for adult onset disorders, will likely need to be given after the patient is already weak from disease. For the muscular dystrophies, this would manifest as impaired motor function due to loss of muscle mass and strength.

Limb Girdle Muscular Dystrophy R9 (LGMDR9; formerly known as LGMD21) is caused by homozygous recessive loss of function mutations in the FKRP gene (Brockington et al., Hum Mol Genet 10:2851-9, 2001), FKRP mutations cause LGMDR ((Brockington et al., Am J Hum Genet 69(6): 1198-209, 2001; Brown et al., Am. J. Pathol. 164(2): 727-37, 2004), and also the more severe Congenital Muscular Dystrophy 1C (MDC1C) (Brockington et al., Hum Mol Genet 10:2851-9, 2001; Brown et al., Am. J. Pathol. 164(2): 727-37, 2004; Longman et al., Hum Mol Genet. 12(21): 2853-61, 2003), by reducing the functional glycosylation of alpha dystroglycan (aDG). Dystroglycan is a member of the dystrophin-associated glycoprotein (DAG) complex, a complex the links ECM proteins through the membrane to the F-actin cytoskeleton inside the muscle cell (Ervasti et al., Cell 66(6): 1121-31, 1992). DG is post-translationally cleaved by an autoproteolytic mechanism to make aDG, an extracellular membrane-associated glycoprotein, and beta dystroglycan (bDG (also known as J3 dystroglycan (J3DG)), a transmembrane glycoprotein (Akhaven et al., FASEB 22(2): 612-21, 2008). Extracellular matrix (ECM) proteins, including laminins that ensheath the myofiber membrane (Ervasti et al., J. Cell Biol 122(4):809-23, 1993; Michele et al. J. Biol. Chem. 278(18): 15457-60, 2003; Michele et al. Nature 418), bind aDG via glycans present in its mucin domain, especially an essential repeating glycosaminoglycan disaccharide made by LARGE (Xylalpha1,3-GlcAbeta1,3) (Inamori et al. Science 335(6064): 93-6, 2012). The creation of this glycan requires at least 18 genes, including FKRP, that help build the glycan substrate on which LARGE acts to complete the glycosylation pathway. In the absence of proper glycosylation, ECM proteins fail to bind to aDG in the muscle membrane, leading to muscular dystrophy. These genetic disorders are collectively referred to as the dystroglycanopathies.

Introduction of AAV.FKRP gene replacement has been shown to recover functional glycosylation of aDG and prevent disease pathology in mouse models of LGMDR9 (Xu et al. Mol Ther, 21(10): p. 1832-40, 2013, Qiao et al. Mol Ther, 2014. 22(11): p. 1890-9, 2014; Awano et al. Am J Pathol, 2015. 185(7): p. 2025-37, 2015, Gicquel et al., Hum Mol Genet. 26(10): p. 1952-1965, 2017). Such therapeutic effects require careful dosing however, as FKRP overexpression can cause dose-dependent pathology (Gicquel et al., Hum Mol Genet. 26(10): p. 1952-1965, 2017). Ribitol and NAD+glycan therapy also has shown therapeutic effects in FKRP mutant mice (Kanagawa et al. J. Neuromuscul Dis. 4(4): 259-267, 2017; Baily et al., Skelet Muscle 9(1): 21, 2019). Supplemental ribitol may increase the concentration of CDP-ribitol, a substrate needed for FKRP glycosylation of aDG with ribitol-6-phosphate (Kanagawa et al. J. Neuromuscul Dis. 4(4): 259-267, 2017). Gene replacement studies in LGMDR9 mouse models clearly show that single FKRP gene therapy is able to prevent loss of ambulation and muscle pathology in young mice, but that it has far less therapeutic effect when given to older mice where disease is already significant (Vannoy et al. Mol Ther Methods Clin Dev 5: 31-42, 2017; Vannoy et al. Mice. Mol Ther Methods Clin Dev 11: 106-120, 2018).

While these proof of concept results for gene replacement are encouraging, LGMDR9 is a very slowly progressive and genetically heterogeneous disease, making testing of therapies that only arrest disease progression quite problematic. MRI studies of LGMDR9 subjects by Volker Straub and colleagues suggest fat replacement of 0-2% of muscle mass per year in 14 different leg muscles (Willis et al., PLoS One 8(8): e70993, 2013). While loss of muscle mass was evident in that study by MRI at one year, no hip, knee, or ankle flexion/extension or adduction/abduction measures, or any walking or time to rise measures, were significantly different when comparing 32 subjects over the same time period (Willis et al., PLoS One 8(8): e70993, 2013) Thus, while gene replacement for these diseases may stop subsequent muscle damage and loss, the slow and variable clinical progression of LGMDR9 makes it very difficult, perhaps impossible, to demonstrate clinically significant improvements in motor function over a 1 year clinical trial period.

Thus, there remains a need in the art for new and effective methods for treating LGMDR9 and MDC1C.

### SUMMARY

Disclosed herein is a bicistronic gene therapy approach that will add a muscle building component to gene replacement. Such therapies have the potential to rebuild loss muscle strength while simultaneously arresting subsequent disease progression. For example, the bicistronic gene therapies disclosed herein were tested using the FKRP_{P448L} mouse model for dystrophy Limb Girdle Muscular Dystrophy R9 (LGMDR9), an adult onset form of muscular dystrophy. This is a knockin mouse model, a model that contains a the P448L human disease mutation (Chan et al., Hum Mol Genet, 2010. 19(20): p. 3995-4006, 2010).

The data provided herein demonstrates that the inclusion of two transcriptional control sequences, e.g. promoters with enhancers, can act synergistically amplify dual transgene expression in skeletal muscles that have muscular dystrophy. Such a gene therapy approach recovered lost walking ability, essentially reversing disease, something single gene replacement therapy has not been shown to do. In addition, the bicistronic (two gene) vectors described herein amplify the level of expression of both therapeutic transgenes relative to the use of a single promoter/enhancer alone. The disclosure provides for a polynucleotide sequence comprising in 5′ to 3′ order: an AAV ITR, a first transcriptional control sequence, an intron, a first transgene sequence, a polyadenylation signal sequence, a second transcriptional control sequence (with promoter, enhancer, and intron), a second (but different) transgene sequence, a second polyadenylation signal sequence, and an AAV ITR.

A transcriptional control sequence includes but are not limited to, promoters, enhancers and/or polyadenylation signal sequences. Examples of transcriptional control sequences include the cytomegaloviruses (CMV promoter), CMV enhancer, miniCMV promoter, MHCK7, CK8 promoter, the chicken p actin promoter, the P546 promoter simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

In an exemplary embodiment, the first transcriptional control sequence is a muscle specific control element. The term “muscle specific control element” refers to a nucleotide sequence that regulates expression of a coding sequence that is specific for expression in muscle tissue. These control elements include enhancers and promoters. The disclosure provides for constructs comprising the muscle specific control elements MHCK7 promoter (Muscle Creatine Kinase promoter (7) w Hybrid intron, the MCK promoter and the MCK enhancer/or alpha myosin heavy chain (MHC) complex enhancer.

Exemplary muscle-specific promoter include one or more of a human skeletal actin gene element, a cardiac actin gene element, a desmin promoter, a skeletal alpha-actin (ASKA) promoter, a troponin I (TNN12) promoter, a myocyte-specific enhancer binding factor MEF binding element, a muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, a murine creatine kinase enhancer element, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE).

In some embodiments, the first transgene is a gene that encodes a protein that is reduced, defective or eliminated in subjects suffering from a degenerative muscular disorder, such as muscular dystrophy. Exemplary transgenes are the Calpain gene 3 (CAPN3), Alpha Sarcoglycan gene (SGCA), Beta Sarcoglycan gene (SGCB), Gamma Sarcoglycan gene (SGCG), delta sarcoglycan gene (SGCD), epsilon sarcoglycan gene (SGCE), Telethionin gene (TCAP), Tripartate motif-containing protein 32 (TRIM32), fukutin-related protein gene (FKRP), fukutin gene (FKTN), Protein 0-mannosyltransferase 1 gene (POMT2), protein 0-mannosyltranferase 2 gene(POMT2), Anoctamin 5 gene (ANO5), Protein O-linked Mannose N-actetylglucyosyltransferase 1 gene (POMGnT1), Dystroglycan gene (DAG1), Desmin gene (DES), GDP-Mannose Pyrophoshorylase B gene (GMPPB), Isoprenoid synthase domain-containing gene (ISPD), Alpha glucosidase gene (GAA), LIM Zinc finger domain containing 2 gene (LIMS2), Blood vessel epicardial structure gene (BVES), Torsin 1A interacting protein gene (TORIA1P1), Protein O-glucosyltransferase 1 gene (POGLUT1), Dysferlin gene (DYSF), Dystrophin gene (DMD), UDP-GIcNAc epimerase/ManNAc 6 kinase gene (GNE), UDP-N-acetyl-galactosaminyltransferase 2 gene (GALGT2 or B4GALNT2), Myotilin gene (MYOT), Lamin A/C gene (LMNA), and Caveolin gene (CAV1).

In an exemplary embodiment, the first transgene is the fukutin-related protein (FKRP) gene. The FKRP gene sequence comprises the nucleotide sequence 3024-4511 of SEQ ID NO: 1 or comprises a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to the nucleotides 3024-4511 of SEQ ID NO: 1 and encodes a functional FKRP protein. The FKRP gene encodes a protein comprising amino acid sequence of SEQ ID NO: 3.

In some embodiments, the second transgene is a gene that encodes a muscle building protein. A muscle building protein is a protein that builds new muscle mass and inducing muscle growth, including a protein that stimulates muscle growth signals or inhibit repressive muscle growth signals. Exemplary transgenes are the follistatin gene (FST), e.g. follistatin 344 (FS344) of follistatin 317 (FS317), Insulin-like growth factor 1 gene (IGF1), heparin binding Epidermal Growth Factor like Growth Factor gene (HB-EGF) or Mothers against decapentaplegic homolog 7 gene (SMAD7).

In an exemplary embodiment, the second transgene is the follistatin (FST) gene that encodes protein form FS344. The FST (FS344) gene sequence comprises nucleotides 5393-6427 of SEQ ID NO: 1 or comprises a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to the nucleotides 5393-6427 of SEQ ID NO: 1 and encodes a functional FS344 protein. The FST (FS344) gene encoded a protein comprising amino acid sequence of SEQ ID NO: 5.

A polynucleotide sequence comprising in 5′ to 3′ order: an AAV ITR, a first transcriptional control sequence operably linked to a nucleotide sequence encoding the FKRP protein, such as a protein comprising the amino acid sequence of SEQ ID NO: 3 and a second transcriptional control sequence operably linked to a nucleotide sequence encoding the FS344 protein, such as a protein comprising the amino acid sequence of SEQ ID NO: 5, and an AAV ITR.

The FKRP gene sequence comprises the nucleotide sequence 3024-4511 of SEQ ID NO: 1 or comprises a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to the nucleotides 3024-4511 of SEQ ID NO: 1 and encodes a functional FKRP protein. The FKRP gene encodes a protein comprising amino acid sequence of SEQ ID NO: 3.

The FS344 gene sequence comprises nucleotides 5393-6427 of SEQ ID NO: 1 or comprises a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to the nucleotides 5393-6427 of SEQ ID NO: 1 and encodes a functional FS344 protein. The FS344 gene encoded a protein comprising amino acid sequence of SEQ ID NO: 5

In any of the polynucleotides disclosed herein, the first transcriptional control sequence is the MHCK7 promoter. For example, the MKCK promoter sequence comprises nucleotides 2056-2846 of SEQ ID NO: 1. Further, in any of the polynucleotides disclosed herein, the second transcriptional control sequence is the CMV promoter and/or CMV promoter. For example, the CMV promoter comprises nucleotides 4591-5117 of SEQ ID NO: 1.

In some embodiments, any of the polynucleotides disclosed herein further comprise a SV40 enhancer and/or an intron, such as a SV40 intron or a chimeric intron. For example, the SV40 enhancer comprises nucleotides 2858-3004 of SEQ ID NO: 1 or comprises nucleotides 5188-5382 of SEQ ID NO: 1. In addition, any of the polynucleotides disclosed herein further comprise a polyadenylation signal sequence, which optionally is a synthetic polyadenylation signal sequence. The polynucleotide sequences disclosed herein comprise an inverted terminal repeat (ITR), such as a mutant ITR or a wild type ITR.

The disclosure also provides for a polynucleotide sequence that is an AAV genome For example, the disclosure provides an AAV genome or polynucleotide sequence comprising a nucleotide sequence that is at least 80%, 81%, 82%, 83%, 84%, 85%, 86%, 87%, 88%, 89%, 90%, 91%, 92%, 93%, 94%, 95%, 96%, 97%, 98% or 99% identical to nucleotides 1847 to 6672 of SEQ ID NO: 1. In addition, the disclosure provides an AAV genome or polynucleotide sequence comprising nucleotides 1847-6672 of SEQ ID NO: 1.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

In addition, the rAAV genomes provided herein, hybridizes under stringent conditions to the polynucleotide sequence of nucleotides 1847 to 2013 of SEQ ID NO: 1 or the complement thereof.

The disclosure also provides for recombinant adeno-associated virus (rAAV) comprising any of the polynucleotide sequences described herein. For example, the rAAV comprises AAV-1, AAV-2, AAV-3, AAV-4, AAV-5, AAV-6, AAV-7, AAV-8, AAV-9, AAV-10, AAV-11, AAV-12, AAV-13, AAVrh.74, AAV rh.10, Anc80, AAV7m8, AAVMyo, MYOAAV capsid protein, or a variant thereof. In some embodiments, the rAAV is a AAVrh.74 serotype. In some embodiments, the rAAV is a AAVMYO3a serotype.

The disclosure also provides for a recombinant AAV particle comprising any of the polynucleotide sequences disclosed herein or any of the rAAV disclosed herein.

In another embodiment, the disclosure provides for methods of producing a rAAV vector particle comprising culturing a cell that has been transfected with any rAAV vector of the disclosure and recovering rAAV particles from the supernatant of the transfected cells. The disclosure also provides for viral particles comprising any of the recombinant AAV vectors of the disclosure.

The disclosure also provides for compositions comprising any of the rAAV disclosed herein or any of the rAAV particles described herein. In some embodiments, the compositions further comprise a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

The disclosure also provides for methods of treating muscular dystrophy comprising administering any of the rAAV disclosed herein or any rAAV particles disclosed herein or any of the compositions disclosed herein to a subject in need thereof. For example, the subject is suffering from limb girdle muscular dystrophy (LGMD), such LGMDR9, or congenital muscular dystrophy 1C (CMD1C). In any of the disclosed methods, the rAAV, rAAV particle or the composition is administered using systemic administration, intramuscular injection or intravenous injection.

The disclosure also provides for compositions for treating muscular dystrophy in a subject in need thereof, wherein the composition comprises any of the rAAV disclosed herein or any rAAV particles disclosed herein or any of the compositions disclosed herein. For example, the subject is suffering from limb girdle muscular dystrophy (LGMD), such LGMDR9, or congenital muscular dystrophy 1C (CMD1C). In any of the disclosed compositions, the rAAV, rAAV particle or the composition is administered using systemic administration, intramuscular injection or intravenous injection.

The disclosure also provides for use of any of the rAAV disclosed herein or any rAAV particles disclosed herein or any of the compositions disclosed herein for the preparation of a medicament for treating muscular dystrophy in a subject in need thereof. For example, the subject is suffering from limb girdle muscular dystrophy (LGMD), such LGMDR9, or congenital muscular dystrophy 1C (CMD1C). In any of the disclosed uses, the rAAV, rAAV particle or the composition is administered using systemic administration, intramuscular injection or intravenous injection.

A “subject,” as used herein, can be any animal, and may also be referred to as the patient. Preferably, the subject is a vertebrate animal, and more preferably the subject is a mammal, such as a domesticated farm animal (e.g., cow, horse, pig) or pet (e.g., dog, cat). in some embodiments, the subject is a human.

### BRIEF DESCRIPTION OF THE DRAWINGS

FIG. 1 provides an annotated plasmid sequence of pAAV.MHCK7.FKRP.spA.CMV.FST(FS344).spA KanR, which is SEQ ID NO: 1.

FIG. 2 demonstrates Induction of muscle mass by FST gene therapy vectors and testing of bicistronic (two promoter), monocistronic, and bicistronic one promoter-IRES containing formulation after IM injection in C57Bl/6J mice provides. Muscles were injected with 1×10¹¹ vg in the tibialis anterior (TA) muscle and with 5×10¹¹ vg in the gastrocnemius (Gastroc) muscle. Errors are SD for n=4 muscles per group. Bicistronic two promoter constructs AAV.MHCK7.FKRP.pA.CMV.FST and AAV.MHCK7.FKRP.pA.Cbh.FST were as good as CMV.FST alone in inducing muscle growth and mass, while other formulations were less good.

FIG. 3 provides AAV biodistribution following intramuscular injection. 2-month-old C57Bl/6J mice were injected with one of several AAV vectors at 1×10¹¹ vg in the tibialis anterior (TA, blue) muscle or 5×10¹¹ vg in the gastrocnemius (Gas) muscle. For AAVMYO3a (Myo3a), 1×10¹⁰ vg was injected in the TA and 5×10¹⁰ vg in the gastrocnemius. Muscles were quantified for the number of AAV vector genomes (vg) per nucleus by qPCR at 4 months of age, 2 months post-injection. TA+Gas represents the average value of both muscles. Errors are SD for n=4 muscles per TA or Gastroc group or n=8 muscles for pooled TA+Gastroc group

FIGS. 4A-4D provide FKRP and FST gene expression after intramuscular (IM) injection of AAV vectors in C57Bl/6J mice. (A,B) Human FKRP gene expression was normalized to endogenous mouse Fkrp gene expression, and (C, D) human FST gene expression was normalized to endogenous mouse Fst gene expression in the tibialis anterior (TA, A,C) and in the gastrocnemius (Gastroc, B,D) muscle. MHCK7.FKRP.pA.CMV.FST here utilized AAV serotype rh74. Errors are SD for n=4 muscles per group. FIG. 5 demonstrates gene expression measures after intramuscular injection of AAV vectors in C57Bl/6J mice. Human FKRP gene expression from the AAV vector, normalized to wild type mouse Fkrp gene expression, and human FST gene expression, relative to endogenous wild type mouse Fst gene expression, were measured by qRT-PCR, referenced to 18S rRNA expression expression, in the tibialis anterior (TA) and in the gastrocnemius (Gastroc) muscles. Errors are SD for n=4 muscles per group. CMV is a stronger promoter than MHCK7.

FIG. 5 demonstrates gene expression measures after intramuscular injection of AAV vectors in C57Bl/6J mice. Human FKRP gene expression from the AAV vector, normalized to wild type mouse Fkrp gene expression, and human FST gene expression, relative to endogenous wild type mouse Fst gene expression, were measured by qRT-PCR, referenced to 18S rRNA expression expression, in the tibialis anterior (TA) and in the gastrocnemius (Gastroc) muscles. Errors are SD for n=4 muscles per group. CMV is a stronger promoter than MHCK7.

FIG. 6 demonstrates IV Treatment with bicistronic AAV in FKRP_{P448L} mice shows complete recovery of uniform normal walking in all subjects in the one our walk test at 15m/min. FKRP_{P448L} mutant (Mut) mice were dosed IV with a high dose (HD, 5×10¹³ vg/kg) or a low dose (LD, 1×10¹³ vg/kg) of monocistronic FKRP (gene replacement), follistatin (FST, muscle building), or Bicistronic (FKRP/FST, Bi) gene therapy. Mut mice showed significantly reduced ambulation relative to WT (p<0.01), while Bi HD showed significantly increased ambulation relative to Mut (PBS), recovering uniformly WT levels (p<0.01). Errors are SD for n=5-15/grp. Statistical analysis Kruskal-Wallis multiple comparisons test, a non-parametric statistical measure.

FIGS. 7A-7B demonstrate maximal tetanic force and force drop during eccentric contractions. In situ force measures in the tibialis anterior muscle of 7 month-old wild type (WT) and FKRP_{P448L} mutant mice were compared. FKRP_{P448L} mice were treated IV with PBS (mock-treated) or with a low dose (LD, 1×10¹³ vg/kg) or a high dose (HD, 5×10¹³ vg/kg) of AAV.CMV.FST, AAV.MHCK7.FKRP, or AAV.MHCK7.FKRP.pA.CMV.FST. Maximal absolute tetanic force (A) and force drop during repeated eccentric contractions (B) are shown. Differences in A are shown only for vs. WT or vs. PBS comparisons. Errors are SD for n=7-20 (A) or 6-18 (B) muscles per group.

FIGS. 8A-8D provide measures of muscle mass, length, cross-sectional area and specific force for in situ physiology studies on the tibialis anterior muscle. Tibialis anterior muscles used in physiology studies were isolated and measured for muscle mass (A), muscle length (B), and cross-sectional area (C), and weight-normalized specific force (D). Errors are SD for n=12-22 muscles per group (A-C) or 720 muscles per group (D).

FIGS. 9A-9B demonstrate muscle hypertrophy after bicistronic FKRP/FST gene therapy. Average Mini-Feret myofiber diameters (in microns) were measured in cross-sections of the TA muscle (A) or the diaphragm (B). Errors are SD for n=9-24 images per group in A and from n=6-22 images per group in B.

FIG. 10 demonstrates induction of muscle mass above wild type level for FST and FKRP/FST gene therapy. Muscle mass was compared to wild type mass for FKRP_{P448L} mice treated at high dose (5×10¹³ vg/kg) with CMV.FST or with MHCK7.FKRP.pA.CMV.FST (FKRP/FST) gene therapy. Errors are SD for n=9-14 muscles per group. Only single muscle FST vs. FKRP/FST comparisons are shown. TA, tibialis anterior, Gastroc, gastrocnemius, Quad, quadriceps femoris, Triceps, triceps brachii.

FIG. 11 demonstrates gene expression measures after IV injection of AAV vectors in FKRP_{P448L} mice. Human FKRP gene expression from the AAV vector, normalized to wild type mouse Fkrp gene expression, and human FST gene expression, relative to endogenous wild type mouse FST gene expression, were measured by qRT-PCR, referenced to 18S rRNA expression. Left column for each muscle group is MHCK7.FKRP or CMV.FST. Right column for each muscle group is MHCK7.FKRP.CMV.FST or MHCK7.FKRP.CMV.FST. Errors are SD for n=6-7 per group. tibialis anterior (TA), gastrocnemius (gastric), quadriceps femoris (quad), triceps brachii (triceps), and diaphragm.

FIG. 12 provides tissue AAV biodistribution after low dose and high dose IV therapy with monocistronic or bicistronic vectors. AAV vector genomes (vg) per cell nucleus were calculated for low dose (LD, 1×10¹³ vg/kg) and high dose (HD, 5×10¹³ vg/kg) IV treatment with AAV.CMV.FST, AAV.MHCK7.FKRP, or AAV.MHCK7.FKRP.pA.CMV.FST gene therapy. Errors are SD for n=5-7 mice per group. TA, tibialis anterior; gastroc, gastrocnemius; quad, quadriceps femoris; tricep, triceps brachii. The order of the columns in each muscle group (from left to right) is: LD AAV.CMV.FST, HD AAV.CMV.FST, LD AAV.MHCK7.FKRP, HD AAV.MHCK7.FKR, LD AAV.MHCK7.FKRP.pA.CMV.FST, HD AAV.MHCK7.FKRP.pA.CMV.FST.

FIGS. 13A-13B demonstrate gene expression changes after high dose monocistronic or bicistronic AAV gene therapy in FKRP_{P448L} mice. IV injections of AAV given at the high dose (5×10¹³ vg/kg) are compared for monocistronic (FKRP or FST) and bicistronic (FKRP/FST) vectors. Left column for each muscle group is MHCK7.FKRP or CMV.FST. Right column for each muscle group is MHCK7.FKRP.CMV.FST or MHCK7.FKRP.CMV.FST. Expression levels were all normalized to endogenous wild type mouse gene expression for that organ and internally referenced to 18S rRNA for each measure. (A) Human FKRP gene expression (hFKRP), normalized to wild type mouse Fkrp (mFkrp) gene expression, is shown. (B) Human FST gene expression (hFST), normalized to wild type mouse Fst gene expression (mFst), is shown. Errors are SD for n=5-7 mice per group in A and B.

FIGS. 14A-14B provide gene expression after low dose IV injection of monocistronic and bicistronic AAV vectors in FKRPP448L mice. IV injections of AAV given at the low dose (1×10¹³ vg/kg) are compared for monocistronic and bicistronic vectors, all normalized to endogenous wild type mouse gene expression for that organ and internal referenced to 18S rRNA for each measure. Left column for each muscle group is MHCK7.FKRP or CMV.FST. Right column for each muscle group is MHCK7.FKRP.CMV.FST or MHCK7.FKRP.CMV.FST. (A) Human FKRP gene expression (hFKRP), normalized to wild type mouse Fkrp (mFkrp) gene expression, is shown. (B) Human FST gene expression (hFST), normalized to wild type mouse Fst gene expression (mFst), is shown. Errors are SD for n=5-6 muscles per group (A, B

FIG. 15 provides expression of follistatin and FKRP protein in liver. Western blots for follistatin (FST) and FKRP protein were done on protein lysates isolated from the liver of wild type (WT) or FKRP_{P448L} mice mock-treated (PBS) or treated with CMV.FST (FST), MHCK7.FKRP (FKRP), or MHCK7.FKRP.pA.CMV.FST (FKRP/FST). GAPDH blotting was done as a control. Each lane is a sample taken from a different mouse.

FIG. 16 provides expression of follistatin and FKRP in skeletal muscle. Western blots for follistatin (FST) and FKRP protein were done on protein lysates isolated from the TA muscle of wild type (WT) or FKRP_{P448L} mice mock-treated (PBS) or treated with CMV.FST (FST), MHCK7.FKRP (FKRP), or MHCK7.FKRP.pA.CMV.FST (FKRP/FST). GAPDH blotting was done as a control. Each lane is a sample taken from a different mouse.

FIGS. 17A-17B provide ELISA measures of serum follistatin levels. Mouse-specific follistatin protein (A) and human-specific follistatin protein (B) were measured by ELISA in serum from wild type mice (WT) or FKRP_{P448L} mice. FKRP_{P448L} mice were either mock-treated (PBS) or treated with low dose (LD, 1×10¹³ vg/kg) or high dose (HD, 5×10¹³ vg/kg) AAV treatments as indicated. Errors are SD for n=4-7 per group in A and B.

FIG. 18 provides western blotting of dystroglycan glycosylation and protein. IIH6, an aDG antibody that recognizes the functional glycan required for laminin binding, and an anti-peptide polyclonal antibody to aDG and bDG protein, were used to blot skeletal protein extracts from the TA muscle of wild type (WT) or FKRP_{P448L} mice. FKRP_{P448L} mice were either mock-treated (PBS) or treated with CMV.FST (FST), MHCK7.FKRP (FKRP), or MHCK7.FKRP.pA.CMV.FST (FKRP/FST). Each FKRP/FST lane represents a muscle from a different mouse dosed with the bicistronic vector. High dose (5×10¹³ vg/kg) treatments only are shown in all instances. Immunoblot for GAPDH antibody is shown as a control for protein loading and transfer.

FIGS. 19A-19D provide quantification of IIH6 immunostaining. Tissue sections from tibialis anterior muscle (A,C) or diaphragm muscle (B,D) were immunostained with IIH6, an antibody that recognizes functional glycosylation of a dystroglycan. The percentage of IIH6-positive myofibers (A,B) and the violin plot of all myofiber pixel intensities for IIH6-positive fibers normalized to the median WT value set at 1 (C,D), were compared in wild type (WT) muscle and in FKRP_{P448L} muscle either mock-treated (PBS) or treated with high dose CMV.FST, MHCK7.FKRP, or MHCK7.FKRP.pA.CMV.FST. Errors are SD for n=10-24 images per group in A and 6-22 images per group mice in B. Violin plots show signal from 6,000-12,000 myofibers per group in C and 6,000-19,000 myofibers per group in D.

FIG. 20 provides percent coincidence of IIH6 with myofiber membrane staining. Pixel coincidence of IIH6 staining with dystrophin staining of muscle membranes was compared in wild type muscle and FKRP_{P448L} muscle mock-treated (PBS) or treated with CMV.FST, MHCK7.FKRP, or MHCK7.FKRP.pA.CMV.FST. Tibialis anterior (TA), diaphragm, and heart are shown. Percent coincidence is color coded from low (blue) to high (red). Non-muscle portions are blocked out of analysis by red lines. Bar is 200 μm for heart and 100 μm for diaphragm and TA panels.

FIG. 21 provides IIH6 staining of wild type and FKRPP448L muscles treated with monocistronic or bicistronic FKRP/FST AAV vectors. IIH6 staining (green, right) and merged dystrophin (red), IIH6 (green), and DAPI (blue) staining (left) was compared in wild type muscle and in FKRPP448L muscles mock-treated (PBS) or treated CMV.FST (FST), MHCK7.FKRP (FKRP), MHCK7.FKRP.pA.CMV.FST (FKRP/FST). Staining with secondary antibody only (2nd only) is shown as a control.

FIGS. 22A-22B provide serum creatine kinase activity before after ambulation protocol. Serum creatine kinase activity was measured before or just after a multi-day ambulation protocol in wild type mice and in FKRP_{P448L} mice mock-treated (PBS) or treated with CMV.FST (FST), MHCK7.FKRP (FKRP), MHCK7.FKRP.pA.CMV.FST (FKRP/FST). Errors are SD for n=2-8 mice (A) or n=4-7(B) mice per group. The order of the columns in each test group (from left to right) for panel A is: WT, PBS, LD AAV.CMV.FST, LD AAV.MHCK7.FKRP, LD AAV.MHCK7.FKRP.pA.CMV.FST. The order of the columns in each test group (from left to right) for panel B is: WT, PBS, HD AAV.CMV.FST, HD AAV.MHCK7.FKRP, HD AAV.MHCK7.FKRP.pA.CMV.FST.

FIGS. 23A-23D demonstrates prevention of muscle damage by monocistronic FKRP and bicistronic FKRP/FST AAV vectors. The percentage of myofibers with central nuclei (A,B) and the percentage non-muscle area (C,D) were measured in the TA (A,C) and diaphragm (B,D) muscle. Errors are SD for 9-24 images per group in A and C and 6-22 images per group in B.

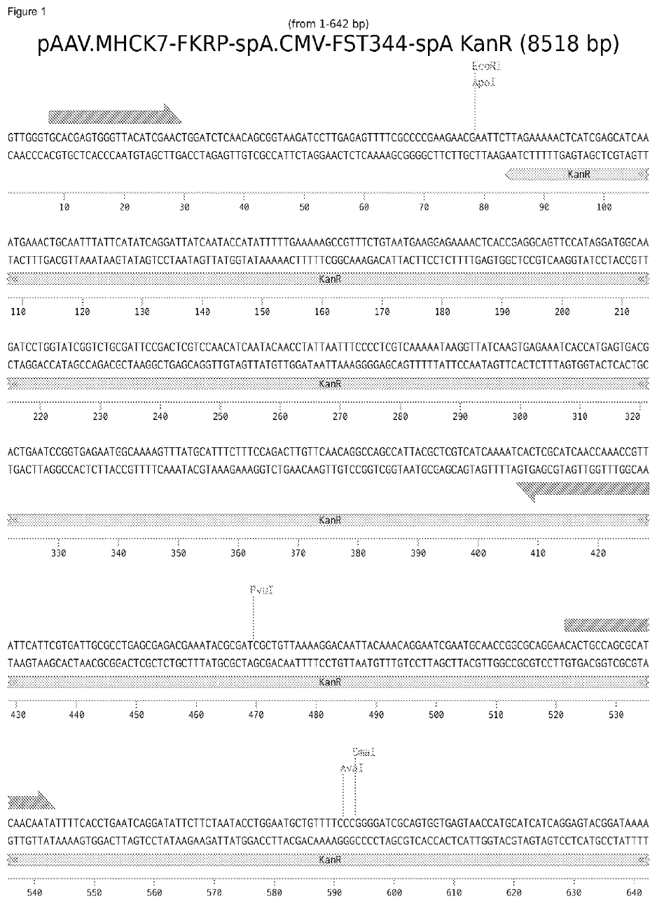

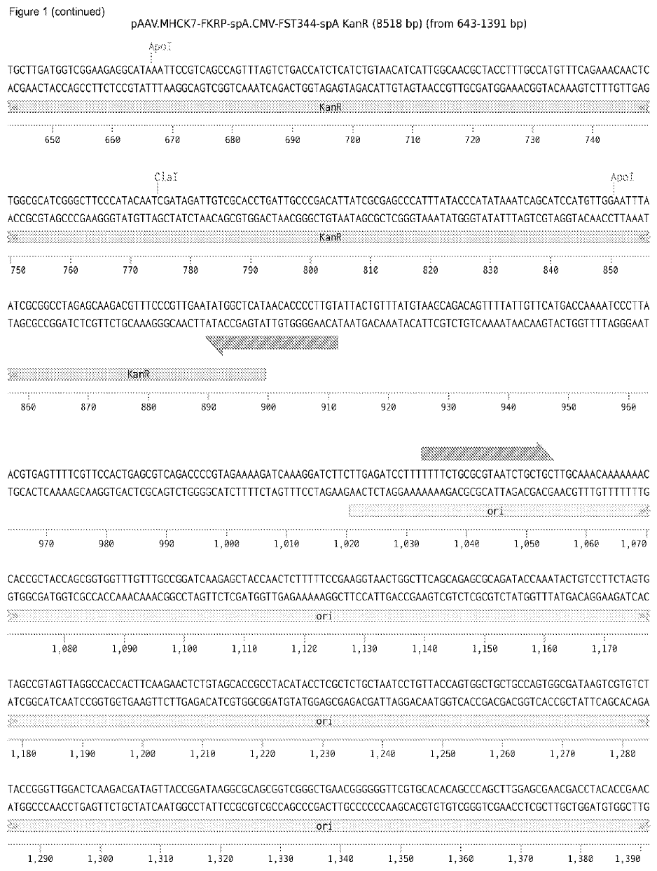

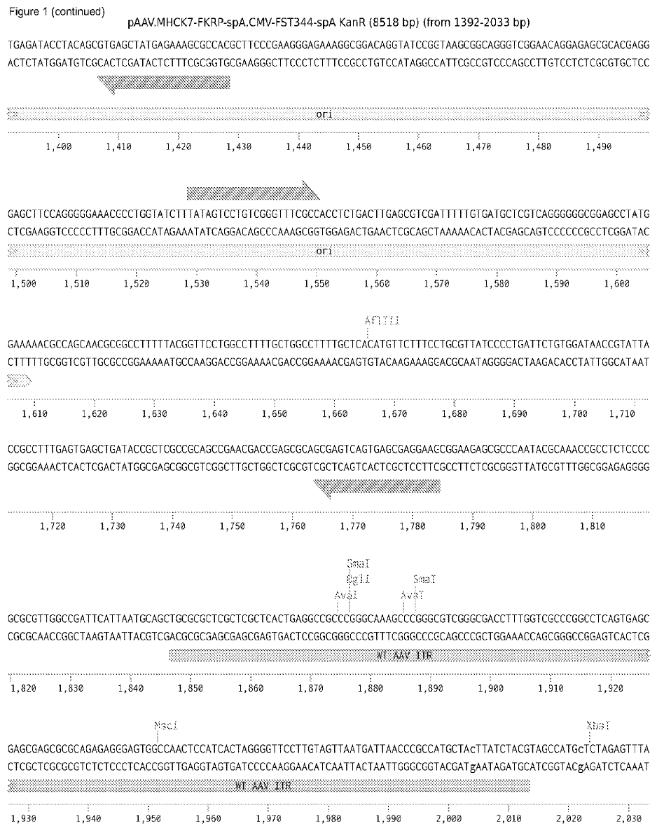

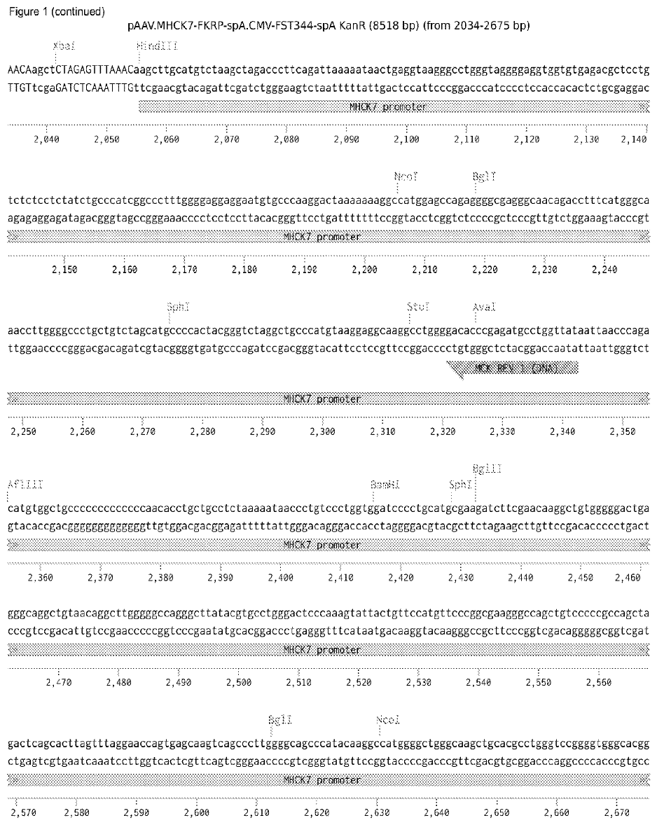

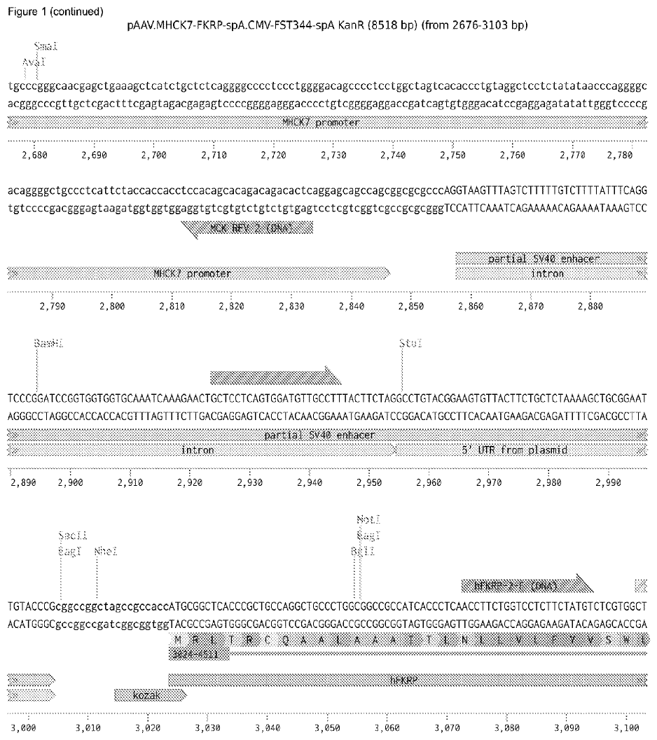

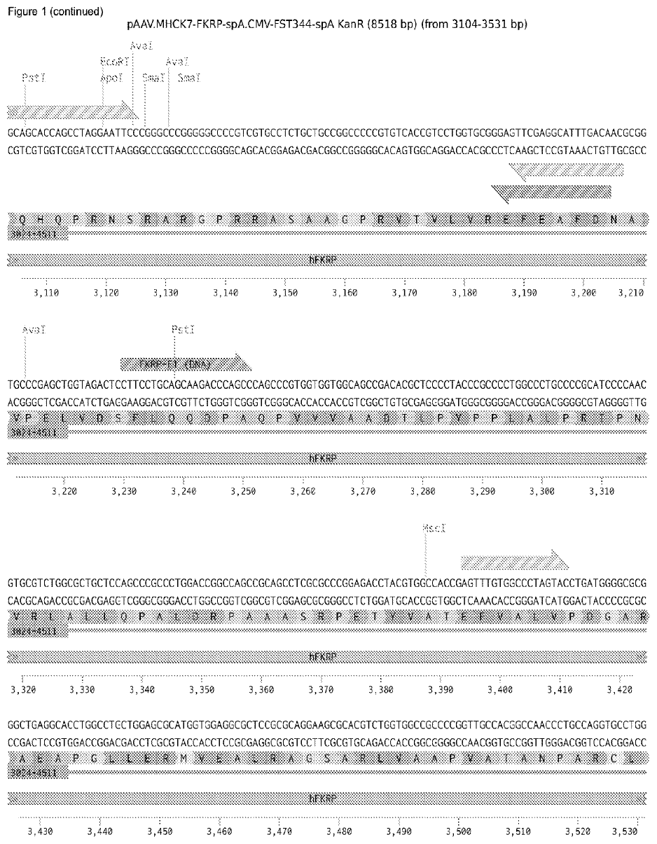

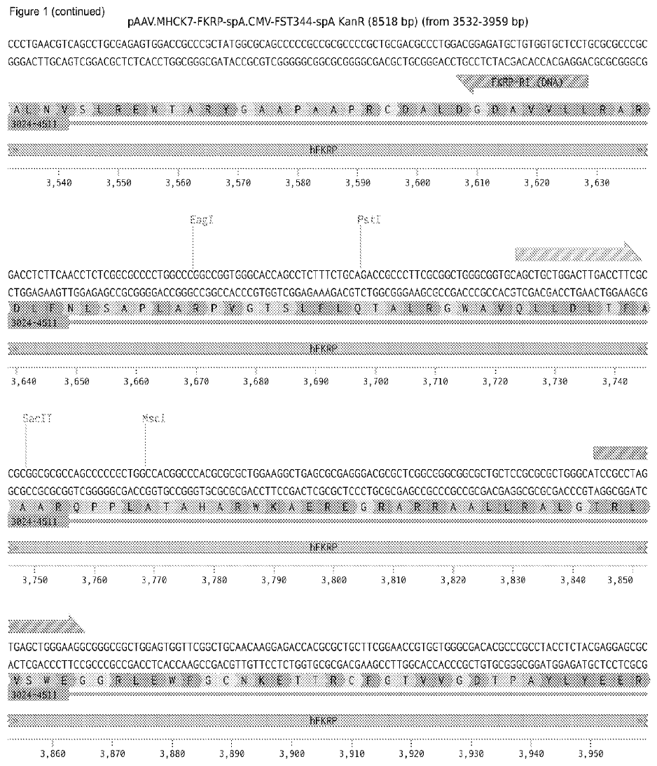

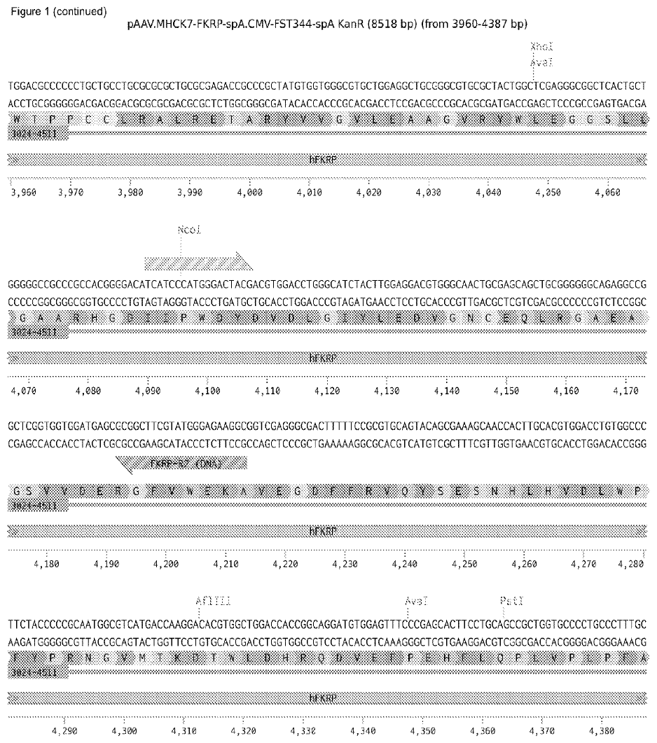

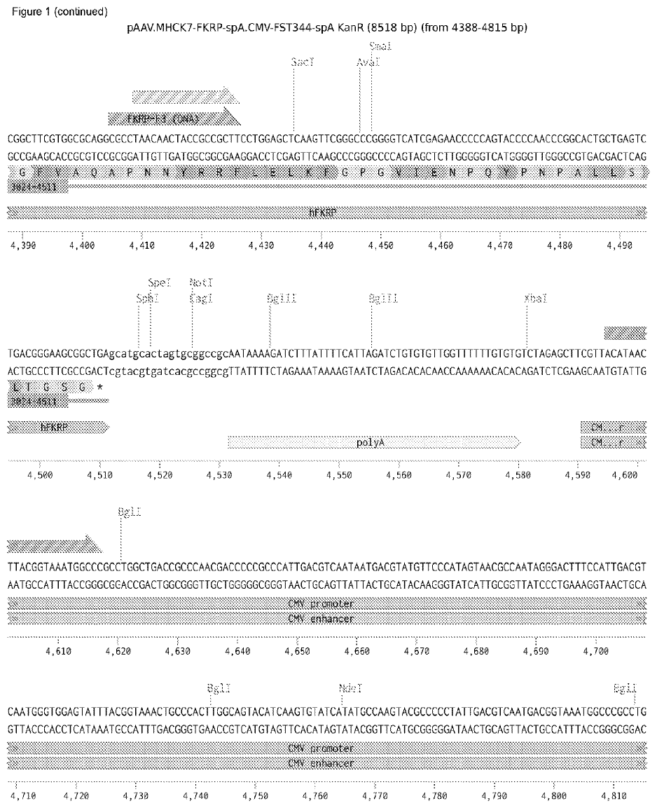

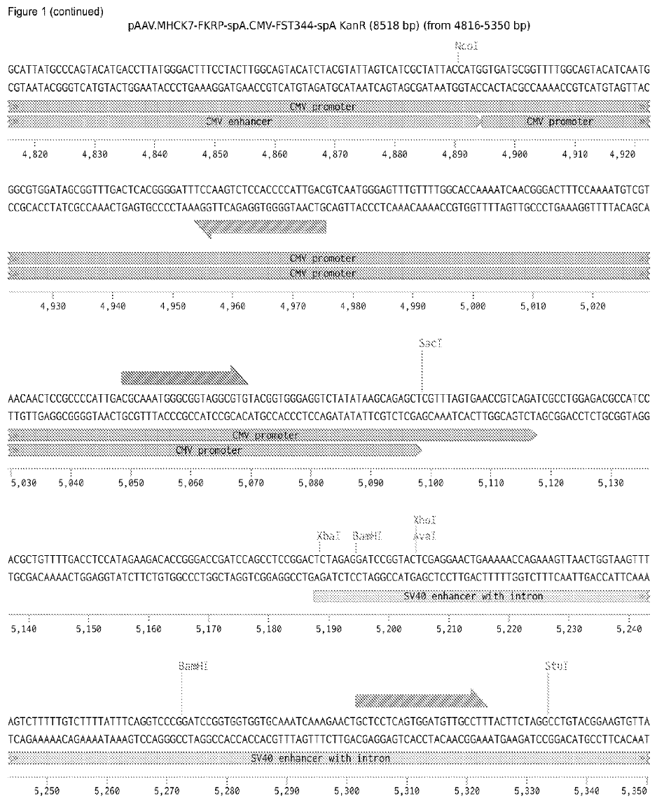

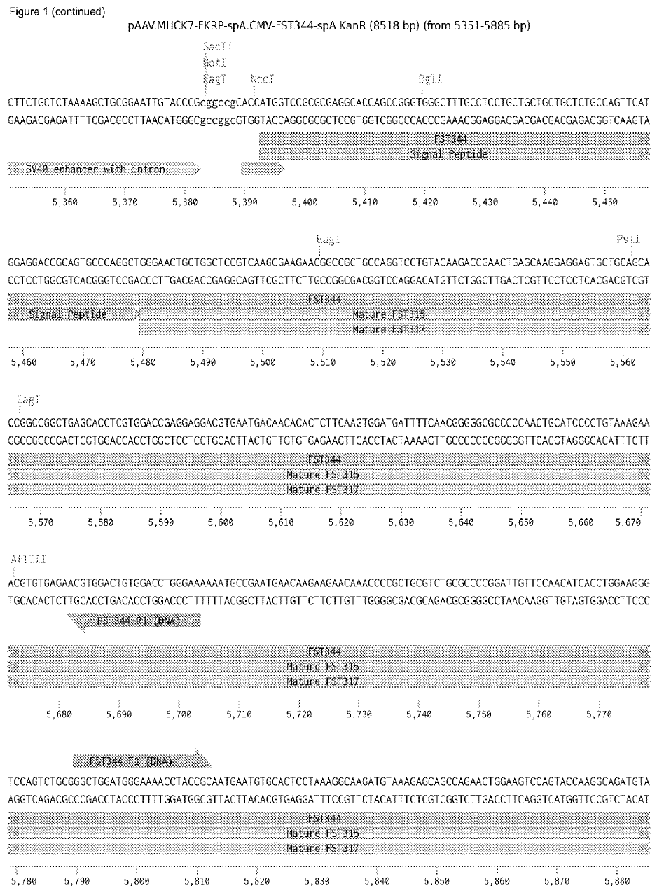

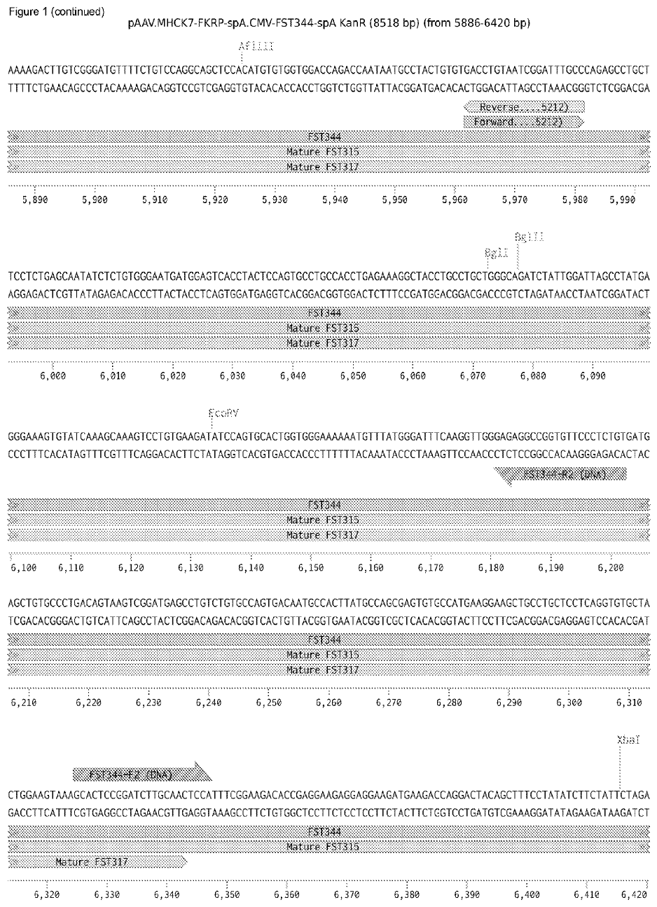

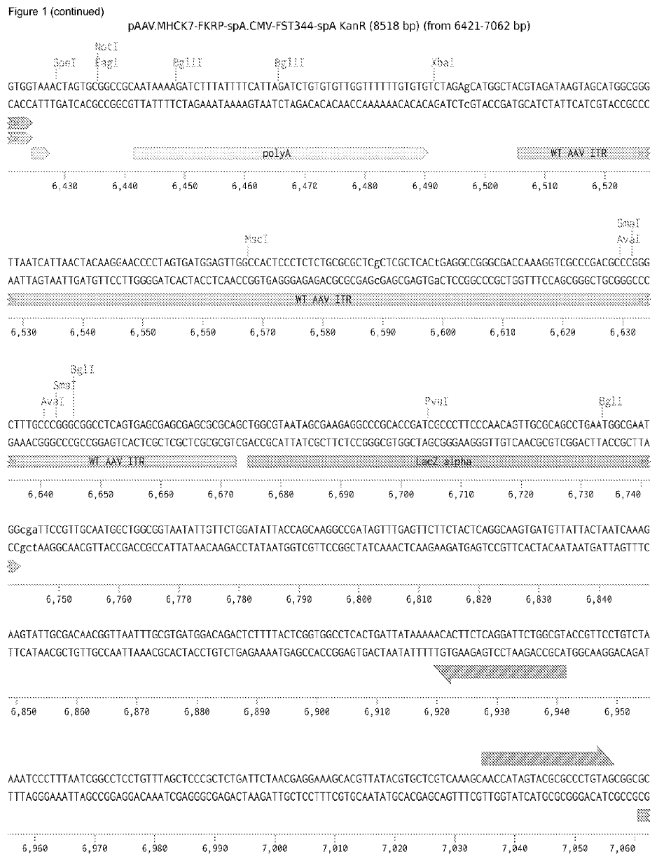

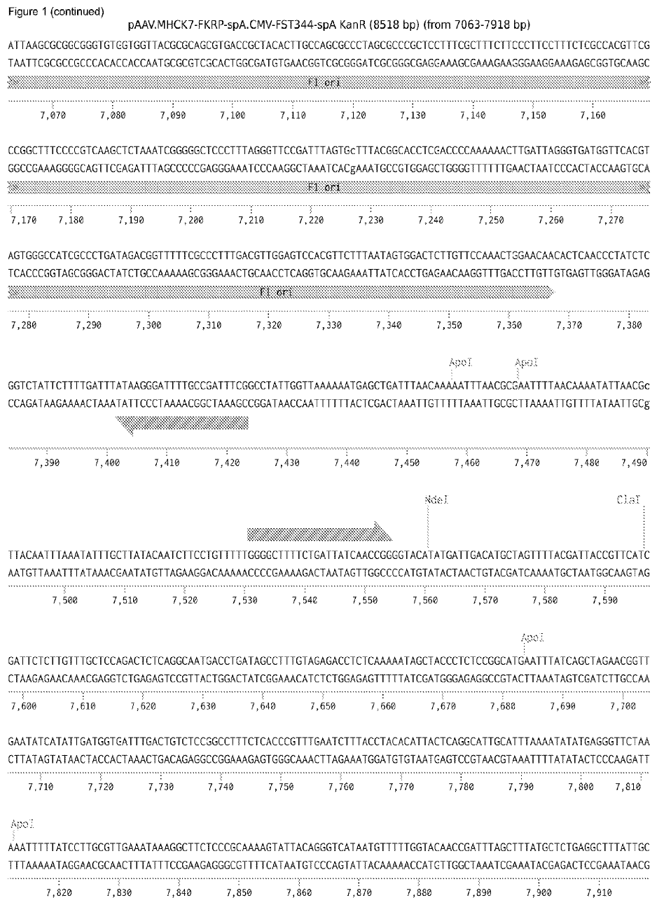

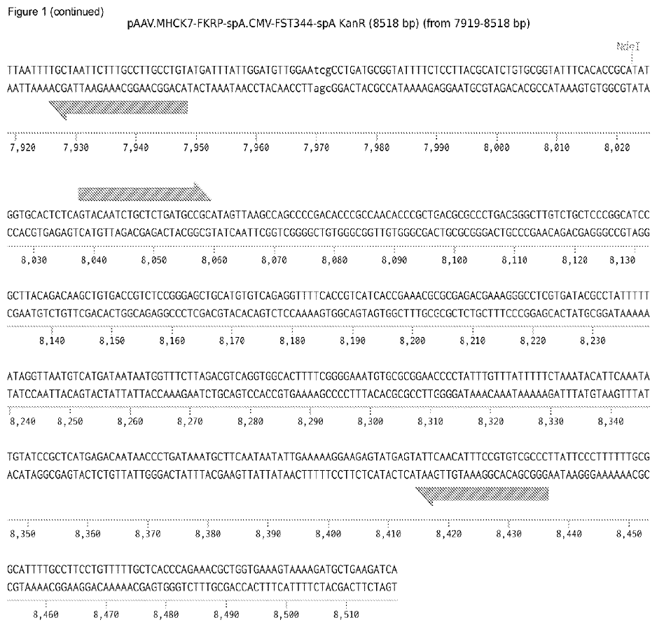

### DETAILED DESCRIPTION

Disclosed herein is a bicistronic gene therapy approach that will add a muscle building component to gene replacement. Such therapies have the potential to rebuild loss muscle strength while simultaneously arresting subsequent disease progression.

To build new muscle mass and strength at the same time as FKRP gene replacement inhibits disease, the disclosed gene therapy approach utilizes follistatin (FST) as a second gene component in bicistronic AAV vectors. FST encodes a secreted myostatin inhibitor protein that binds to and inhibits myostatin protein binding to muscle cells (Amthor et al., Dev Biol, 270(1): p. 19-30, 2004). Myostatin, is a secreted muscle trophic factor that negatively regulates muscle growth and strength. Elimination of myostatin in mice, cows, or humans can double the size of skeletal muscles, with minimal to no effects on cardiac muscle or non-muscle tissues. Elimination of myostatin in mdx mice significantly increased muscle size and strength, but did not improve weight-normalized grip strength or specific (weight-normalized) tetanic muscle force. Thus, myostatin inhibition does not stabilize the muscle membrane or prevent muscle damage, but instead increases muscle strength by increasing muscle mass. In this sense then, myostatin inhibition therapy alone may be ineffective over the long term, as muscles expressing the inhibitor will eventually be destroyed if effective gene replacement is not also provided. Indeed, a recent clinical trial of myostatin inhibition therapy, using a blocking antibody approach, failed to meet any of its clinical milestones in an LGMDR9 clinical trial (Leung et al., Muscle Nerve, 2021. 64(2): p. 172-179, 2021).

The FST gene form used in the exemplary approach described herein, FS344, has been tested in two clinical trials: IM delivery of rAAV1.CMV.FST bilaterally in the quadriceps muscles of patients with Becker Muscular Dystrophy (BMD) yielded improvements in the 6-minute walk test (6MWT, as much as 125 meters) in 4 of 6 patients at 1 year post-treatment. Treated BMD patient muscle biopsies suggested muscle hypertrophy, decreased endomysial fibrosis, and more uniform myofiber size (Mendell et al., Mol Ther, 23(1): p. 192-201, 2015). Similar IM delivery of rAAV1.CMV.FST in patients with Inclusion Body Myositis (IBM) patients yielded an average improvement of +56 meters on the 6MWT at one year post-treatment (and as high as +153 m) in six subjects. Treated IBM patient muscle biopsies showed muscle hypertrophy, decreased fibrosis, and improved regeneration (Mendell et al., Mol. Ther. 23(1): 192-201, 2015). Thus, a major attraction to the use of FST gene therapy in bicistronic vectors is that this gene has already been tested in humans and shown to be safe and effective. Another major attraction is that FST gene therapy is more potent than myostatin inhibition alone (Lee et al., PLoS One 2(8): e789, 2007) and can activate additional muscle growth signaling pathways (Winbanks et al., J. Cell Biol. 197(7):997-1008, 2012).

In particular, the data provided herein provides evidence that coupling gene replacement (FKRP) to a second gene known to build muscle mass and strength (FST) can recover lost muscle function and build new muscle strength in the FKRP_{P448L} mouse model of LGMDR9. In fact, this gene therapy builds muscle strength in treated FKRP_{P448L} mice beyond that normally found in wild type mice. FST built new muscle mass and strength both by inducing the muscle cell growth (hypertrophy) and new muscle cell formation (hyperplasia). The use of a constitutive promoter (CMV) to drive FST expression in a bicistronic FKRP/FST AAV elevated serum protein levels as high as 11-fold above normal endogenous levels. This may have allowed for induction of muscle growth in trans in cells where the gene therapy was not present.

The use of the more muscle-specific MHCK7 promoter to drive FKRP expression simultaneously allowed for induction of functional glycosylation on a dystroglycan (aDG). Defective functional glycosylation of aDG is the molecular defect that causes disease in LGMDR9. FKRP/FST therapy not only increased the number of muscles cells with aDG glycosylation but it increased the intensity and coincidence of such glycosylation along myofiber membranes. This again exceeded levels found in wild type muscle. MHCK7 expression also allowed for some non-muscle expression of FKRP; FKRP protein was identified in western blots of liver protein. This may in part reflect the large induction of FKRP gene expression with the bicistronic vector.

The data provided herein suggests that engineering both genes into a single AAV vector has distinct advantages. One advantage is that both genes will be assured of delivery to the same cells in every instance. A second advantage is that treatment will require half the AAV dose to do so. The data provided herein show additional unexpected advantages of the bicistronic AAV. First, use of a bicistronic AAV amplified gene expression for both transgenes compared to single AAVs alone using the same promoter. While some of this increase resulted from loss of single FST AAV vector due to muscle damage, some of the effect was due to higher induction of transcription resulting from the presence of two promoters, where the distinct enhancers from each of those promoters likely acted to amplify the expression of both genes. Second, bicistronic FKRP/FST had more profound effects on muscle growth than single FST vector alone. Though FKRP/FST and FST induced similar amounts of muscle growth in some limb muscles (triceps, quad, TA), FKRP/FST doubled muscle growth in the gastrocnemius relative to FST alone, keeping the induction of growth in this muscle more on a par with induced growth found in the other limb muscles. More even muscle growth may prevent contractures and provide more effective limb muscle counterbalances that would improve ambulation. FKRP/FST also significantly increased the size of muscle fibers in the diaphragm, while FST alone did not. Given that recent studies have implicated reduced respiratory capacity as a phenotype in FKRPP448L mice ( ), improved diaphragm strength may also contribute to improved ambulation endurance.

Another important, perhaps underappreciated, aspect of bicistronic therapy is that it may expand the patient groups that can participate in clinical trials. If disease prevention is all that is anticipated, only patients with matched disease can be used to effectively assess therapeutic efficacy. Ideally such a patient cohort would comprise only young patients that are not already severely affected by disease. LGMDR9 is a slowly progressive and genetically heterogeneous disease. MRI studies of LGMDR9 subjects by Straub and colleagues showed fat replacement of muscle mass at a rate of 0-2% per year for 14 different leg muscles (Xiao et al. J Virol 72: 2224-2232). While loss of muscle mass was evident in that study at one year by MRI, no hip, knee, or ankle flexion/extension or adduction/abduction measures, or any walking or time to rise measures, were significantly different when comparing 32 subjects over the same time period (Willis et al., PLoS One 8: e70993, 2013). Thus, the slow and variable clinical progression of LGMDR6 makes it very difficult, perhaps impossible, to demonstrate clinically significant improvements in motor function over the typical 1-year period commonly used for clinical trials. The ability of bicistronic vectors to improve strength beyond baseline should allow patients with different baseline strengths to be compared to themselves post-treatment. This should open up clinicals to include more variably and more severely affected patients, where such bicistronic treatments may still yield clinical improvements.

### Limb Girdle Muscular Dystrophies

Limb girdle muscular dystrophies (LGMDs) are rare conditions and they present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, people can have problems with walking and may need to use a wheelchair over time. The involvement of shoulder and arm muscles can lead to difficulty in raising arms over head and in lifting objects. In some types of LGMD, the heart and breathing muscles may be involved.

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMD1E | Autosomal dominant | Desmin gene |
| LGMD1F | Autosomal dominant | Chromosome 7 |
| LGMD1G | Autosomal dominant | Chromosome 4 |
| LGMD1H | Autosomal dominant | Chromosome 3 |
| LGMD2A | Autosomal recessive | Calpain-3 gene |
| LGMD2B | Autosomal recessive | Dysferlin gene |
| LGMD2C | Autosomal recessive | Gamma-sarcoglycan |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMDR9 | Autosomal recessive | FKRP gene |
| LGMD2J | Autosomal recessive | Titin gene |
| LGMD2K | Autosomal recessive | POMT1 gene |
| LGMD2L | Autosomal recessive | Anoctamin 5 gene |
| LGMD2M | Autosomal recessive | Fukutin gene |
| LGMD2N | Autosomal recessive | POMT2 gene |
| LGMD2O | Autosomal recessive | POMGnT1 gene |
| LGMD2Q | Autosomal recessive | Plectin gene |

Specialized tests for LGMD are now available through a national scheme for diagnosis, the National Commissioning Group (NCG).

The provided gene therapy is useful for treating limb girdle GNE myopathies, Duchenne and Becker muscular dystrophies (DMD and BMD) and limb girdle muscular dystrophies (LGMD) such as LGMD2A (CAPN3, LGMDB (DYSF), LGMD2C (SGCG), LGMD2D (SGCA), LGMD2E (SGCB), LGMD2F (SGCD), LGMD2G (TCAP), LGMD2H (TRIM32), LGMDR9 (FKRP), LGMD2J (TTN), LGMD2K (POMT1), LGMD2L (ANO5), LGMD2M (FKTN), LGMD2N (POMT2), LGMD20 (POMT2), LGMD2P (DAG1), LGMD2Q PLEC1), LGMD2R (DES), LGMD2S (TRAPPPCII), LGMD2T (GMPPB) LGMD2U (ISPD), LGMD2V (GAA), LGMD2X (BVES), LGMD2Y (TOR1AIP1), LGMD2Z (POGLUT1), LGMD1A (TTID, MYOT), LGMD1B (LMNA), LGMD1C (CAV3), LGMD1D (DES), LGMD1F (TNPO3), and LGMD1G (HNRPDL). In each instance, the first transgene may be used for gene replacement for the gene missing in the disease or a surrogate gene replacement, while the second transgene encodes a muscle building protein growth factor such as FS344, FS315, FS317, FS288, HB-EGF1, IGF1 or SMAD7 to reverse disease symptoms by building new muscle growth and strength.

### Muscle Building Proteins

Muscle building proteins can include growth factors that induce muscle growth or increase muscle strength such as IGF, HB-EGF, Pax7, HGF (hepatocyte growth factor), HGH (human growth hormone), FGF19 (fibroblast growth factor 19), FGF21 (fibroblast growth factor 21), VEGF (vascular endothelial growth factor), IL6 (Interleukin 6), IL15 (Interleukin 15) and SMAD7 (mothers against decapentaplegic homolog 7 (MADH7)).

Growth factors that induce muscle growth or increase muscle strength also include the follistatins (FST). Follistatin is a secreted protein that inhibits the activity of TGF-β family members such as GDF-11/BMP-11. Follistatin-344 is a follistatin precursor that undergoes peptide cleavage to form the circulating Follistatin-315 isoform which includes a C-terminal acidic region. It circulates with myostatin propeptide in a complex that includes two other proteins, follistatin related gene (FLRG) and GDF associated serum protein (GASP-1). Follistatin-317 is another follistatin precursor that undergoes peptide cleavage to form the membrane-bound Follistatin-288 isoform.

The DNA and amino acid sequences of the follistatin-344 precursor are respectively set out in SEQ ID NOs: 4 and 5 (and the nucleotides 5393-6427 of SEQ ID NO: 1). FS344 contains a C-terminal protein domain lacking in FS288. The presence of this C-terminal domain reduces binding to activin and to heparan sulfate glycosaminoglycans, which in turn reduces non-muscle effects. The Follistatin-288 isoform, which lacks a C-terminal acidic region, exhibits strong affinity for heparin-sulfate-proteoglycans, is a potent suppressor of pituitary follicle stimulating hormone, is found in the follicular fluid of the ovary, and demonstrates high affinity for the granulose cells of the ovary. The testis also produce Follistatin-288. Lack of follistatin results in reduced muscle mass at birth.

Examples of follistatins are provided in Shimasaki et al., U.S. Pat. No. 5,041,538, other follistatin-like proteins are provided in U.S. Pat. Nos. 5,942,420; 6,410,232; 6,537,966; and 6,953,662), FLRG is provided in Hill et al., J. Biol. Chem., 277(43): 40735-40741 (2002)] and GASP-1 is provided in Hill et al., Mol Endocrinol, 17: 1144-1154 (2003).

SMAD7 is known to inhibit TGF-β-activated signaling responses by associating with the active TGF-β complex, which results in reduced TGF-β signaling. Myostatin and TGF-β signaling induces SMAD7 expression establishing a negative feedback loop to inhibit TGF-β signaling. In particular, SMAD7 is known to modulate myogenesis using this negative feedback loop (Kollias et al. Mol. Cell Biol. 26(16):6248-6260, 2006). The nucleotide sequence encoding the SMAD7 is provided in Genbank Accession No. NM_005904.4, and the amino acid sequence is provided as Genbank Accession No. NP_005895.

Transdifferentiation factors are agents that convert or induce differentiation to a non-muscle cell to muscle. For example, MyoD is known to convert a number of cell types into muscle, including dermal fibroblasts, chondrocytes, smooth muscle, retinal pigmented epithelial cells, adipocytes, and melanoma, neuroblastoma, osteosarcoma, and hepatoma cells (Abraham & Tapscott, Curr. Opin. Genet. Dev. 23(5): 568-573, 2013). Other examples of transdifferentiatation factors Myocd (myocardin), Mef2C (myocyte enhancer factor 2C), Mef2B (myocyte enhancer factor 2B), Mkl1 (MKL [megakaryoblastic leukemia]/Myocd-like 1), Gata4 (GATA-binding protein 4), Gata5 (GATA-binding protein 5), Gata6 (GATA-binding protein 6), Ets1 (E26 avian leukemia oncogene 1, 5′ domain).

### AAV Gene Therapy

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including an inverted terminal repeat (ITRs). Exemplary ITR sequences may be 130 base pairs in length or 141 base pairs in length, such as the ITR sequence. There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8, AAVMyo, MYOAAV, and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12, AAVrh.10, AAV-13, Anc80, AAV7m8, AAVMyo or MYOAAV. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising rAAV of the present disclosure. Compositions of the disclosure comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Titers and dosages of rAAV to be administered in methods of the invention will vary depending, for example, on the particular rAAV, the mode of administration, the treatment goal, the individual, the timing of administration, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). These dosages of rAAV may range from about 1×10⁹ vg or more, about 1×10¹⁰ vg or more, about 1×10¹¹ vg or more, about 1×10¹² vg or more, about 6×10¹² or more, about 1×10¹³ vg or more, about 1.3×10¹³ vg or more, about 1.4×10¹³ vg or more, about 2×10¹³ vg or more, about 3×10¹³ vg or more, about 6×10¹³ vg or more, about 1×10¹⁴ vg or more, about 3×10¹⁴ or more, about 6×10¹⁴ or more, about 1×10¹⁵ vg or more, about 3×10¹⁵ or more, about 6×10¹⁵ or more, about 1×10¹⁶ or more, about 3×10¹⁶ or more, or about 6×10¹⁶ or more. For a neonate, the dosages of rAAV may range from about 1×10⁹ vg or more, about 1×10¹⁰ vg or more, about 1×10¹¹ vg or more, about 1×10¹² vg or more, about 6×10¹² or more, about 1×10¹³ vg or more, about 1.3×10¹³ vg or more, about 1.4×10¹³ vg or more, about 2×10¹³ vg or more, about 3×10¹³ vg or more, about 6×10¹³ vg or more, about 1×10¹⁴ vg or more, about 3×10¹⁴ or more, about 6×10¹⁴ or more, about 1×10¹⁵ vg or more, about 3×10¹⁵ or more, about 6×10¹⁵ or more, about 1×10¹⁶ or more, about 3×10¹⁶ or more, or about 6×10¹⁶ or more.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Example of a disease contemplated for prevention or treatment with methods of the disclosure is limb girdle muscular dystrophy, e.g. LGMDR9, or congenital muscular dystrophy 1C.

The term “transduction” is used to refer to the administration/delivery of the coding region of the transgenes, e.g. FKRP gene and FST gene, to a recipient cell either in vivo or in vitro, via a replication-deficient rAAV of the disclosure resulting in expression of FKRP protein and FST protein in the recipient cell.

Transduction of cells with rAAV of the disclosure results in sustained expression of the proteins encoded by the first and second transgenes. The present disclosure thus provides methods of administering/delivering rAAV to an animal, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one embodiment of the disclosure provides methods of transducing muscle cells and muscle tissues directed by muscle specific promoter elements, including, but not limited to, those derived from the actin and myosin gene families, such as from the myoD gene family (See Weintraub et al., Science, 251: 761-766 (1991)), the myocyte-specific enhancer binding factor MEF-2 (Cserjesi and Olson, Mol Cell Biol 11: 4854-4862 (1991)), control elements derived from the human skeletal actin gene (Muscat et al., Mol Cell Biol, 7: 4089-4099 (1987)), the cardiac actin gene, muscle creatine kinase sequence elements (See Johnson et al., Mol Cell Biol, 9:3393-3399 (1989)) and the murine creatine kinase enhancer (MCK) element, MHCK7, control elements derived from the skeletal fast-twitch troponin C gene, the slow-twitch cardiac troponin C gene and the slow-twitch troponin I gene: hypoxia-inducible nuclear factors (Semenza et al., Proc Nat Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the disease state being treated and the target cells/tissue(s) that are to express the protein encoded by the first and/or second transgene.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include sirolimus, everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus.

Purine analogs block nucleotide synthesis and include IMDH inhibitors. Exemplary purine analogs include azathioprine, mycophenolate and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immunosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

The following EXAMPLES are provided by way of illustration and not limitation. Described numerical ranges are inclusive of each integer value within each range and inclusive of the lowest and highest stated integer.

### EXAMPLES

### Example 1

### Construction of Bicistronic AAV Vector

The disclosed plasmid of pAAV.MHCK7.FKRP.spA.CMV.FST344.spA is set out as SEQ ID NO: 1. This plasmid contains an expression cassette flanked by AAV2 inverted terminal repeat sequences (ITR), these expression cassettes may also comprise a first transcriptional control sequence, MHCK7 operably linked to the transgene encoding the FKRP protein and a second transcriptional control sequence, CMV promoter/enhancer operably linked a transgene encoding FST344.

Human FKRP (NM_001039885.3) cDNA was synthesized by Twist Biosciences (San Francisco, CA) with 5′ Nhel/3′ Sphl-Spel-Notl restriction enzyme cut sites and a 5′ Kozak consensus sequence (GCCGCCACCATG). Human FST (NM_013409.3) cDNA was synthesized by GeneArt (Thermo Fisher; Waltham, MA) with 5′ and 3′ Notl restriction enzyme cut sites and a 5′ Kozak consensus sequence (ACCATGG). FKRP, FST, or SMAD7, or GFP were cloned into pAAV vectors under the various promoters, including MCK (CK7-like), MHCK7, CMV, or CBH, with or without the internal ribosomal entry site (IRES) from FGF1 (Dulluc-Clavieres et al. Gene Ther 15: 1090-1098, 2008; Gray et al. Hum Gene Ther 22: 1143-1153, 2011; Xu et al., Mol Ther Methods Clin Dev 10: 89-104, 2011; Salva et al., Mol Ther 15: 320-329, 2007).

The bicistronic expression cassette had a Kanamycin resistance gene, and an optimized Kozak sequence, which allows for more accurate and robust protein translation. rAAV vectors were produced by a modified cross-packaging approach whereby the AAV type 2 vector genome can be packaged into multiple AAV capsid serotypes (Rabinowitz et al., J Virol. 76 (2):791-801 (2002)). Production was accomplished using a standard three plasmid DNA/CaPO4 precipitation method using HEK293 cells. HEK293 cells were maintained in DMEM supplemented with 10% fetal bovine serum (FBS) and penicillin and streptomycin. The production plasmids were: (i) plasmids encoding the therapeutic proteins, (ii) rep2-capX modified AAV helper plasmids encoding cap serotype AAVrh74 isolate, and (iii) an adenovirus type 5 helper plasmid (pAdhelper) expressing adenovirus E2A, E4 ORF6, and VA I/II RNA genes. The AAV were purified using iodixanol density centrifugation and anion exchange chromatography (Clark et al., Human gene therapy 10: 1031-1039, 1999) A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg ml⁻¹) was determined by quantitative reverse transcriptase PCR using the specific primers and probes utilizing a Prism 7500 Real-time detector system (PE Applied Biosystems, Grand Island, NY, USA). Aliquoted viruses were kept at −80° C. until production.

All plasmids used to make AAV genomes to be packaged also contain a Kanamycin resistance gene (KanR) outside of the ITR sequences used for packaging of the genome. This allows for the DNA encoding the AAV genome to be transformed into bacteria to produce large amounts of DNA in the presence of Kanamycin, which will kill all non-transformed bacteria. KanR is not packaged into the AAV capsid in the AAV genome used to treat patients, but its presence allows for DNA production in bacteria.

A description of pAAV.MHCK7.FKRP.spA.CMV.FST344.spA is set out as SEQ ID NO: 1 is provided in the Table below.

| | Nucleotides in SEQ ID | |
|---|---|---|
| Element | NO: 1 | Direction |
| KanR | 83-899 | [symbol] |
| Ori | 1021-1609 | [symbol] |
| Wild type AAV ITR | 1847-2013 | |
| MHCK7 promoter | 2056-2846 | [symbol] |
| Partial SV40 | 2858-3004 | [symbol] |
| enhancer/intron | | |
| Kozak | 3016-3026 | [symbol] |
| Human FKRP cDNA | 3024-4511 | [symbol] |
| Poly A | 4532-4580 | [symbol] |
| CMV promoter/enhancer | 4591-5117 | [symbol] |
| SV40 enhancer with intron | 5188-5382 | [symbol] |
| FST344 cDNA | 5393-6426 | [symbol] |
| Poly A | 6442-6491 | [symbol] |
| Wild type AAV ITR | 6506-6672 | |
| LacZ alpha | 6675-6743 | [symbol] |
| F1 ori | 7061-7367 | [symbol] |

### Example 2

### Screening of Bicistronic AAVs for Increased Muscle Mass in Wild Type Mice

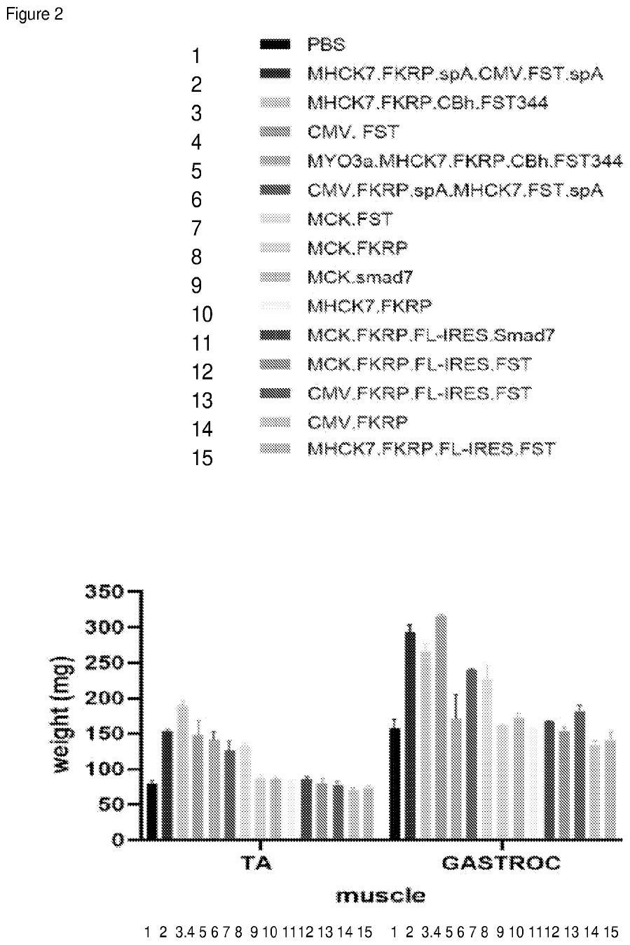

Various bicistronic (two gene) formulations were screened to identify those that would best utilize follistatin to induce muscle growth when it was placed in the second position of an AAV gene therapy vector. Wild type C57Bl/6J mice were injected at 2 months of age intramuscularly with 1×10¹¹ vg (vg is vector genomes) of AAV in the tibialis anterior (TA) muscle and with 5×10¹¹ vg in the gastrocnemius muscle. Mice were euthanized 2 months after injections and muscles weighed to determine increased muscle mass. Muscles were dissected from tendon to tendon and weighed to determine increased muscle mass (FIG. 2). Two types of vectors were compared. In one set, we utilized a single promoter and placed an internal ribosome entry site (IRES) from FGF1A, an element known to work well in skeletal muscles (Delluc-Clavieres et al. Gene Therp. 15(15): 1090-8, 2008), to drive expression of the second follistatin protein. We had previously characterized this IRES element and shown its ability to drive GFP expression in non-muscle cells (ns).

The second configuration compared placement of two promoters to separately drive mRNA transcripts for each gene, with a polyA placed at the end of each cDNA. Here we compared use of a strong constitutive promoter (either cytomegalovirus (CMV) or chicken beta actin with hybrid intron (Cbh)) or a strong muscle-specific promoter (muscle creatine kinase (type 7-like) (MCK) or MCK with a hybrid enhancer (MHCK7)). In all instances, the human FKRP gene was placed in the first position of the bicistronic vector and the human follistatin (FST) gene, form FS344 (Rodino-Klapac et al. Muscle Nerve 39(3): 283-96, 2009), was placed in the second position. These were compared to monocistronic constructs where only a single gene was expressed. With two exceptions, all AAVs were produced using the rhesus 74 (rh74) serotype, a serotype that has performed well with regard to muscle transduction in DMD clinical trials (Mendell et al. JAMA Neurol. 77(9): 1122-1131, 2020). rAAV.CMV.FST utilized AAV9, which also transduces muscle well based on our previous studies (Rodino-Klapac et al. Muscle Nerve 39: 283-296, 2009), and AAV.MHCK7.FKRP.pA.CBH.FST utilized either AAVrh74 or AAVMYO3a (Myo3a). Because AAVMYO3a it about 10 times more myotrophic than rAAV9 (Weinmann et al., Nat Commun 11: 5432, 2020), rAAVMyo3a was injected at a 10th the dose of the other vectors.

AAV.CMV.FST doubled the size of the TA or the gastroc muscle within the two months of treatment in these experiments. For bicistronic formulations, AAV.MHCK7.FKRP.pA.CMV.FST and AAV.MHCK7.FKRP.pA.Cbh.FST induced as much growth as AAV.CMV.FST, with CMV showing slightly better results than Cbh. Use of rAAVMYO3a for rAAV.MHCK7.FKRP.pA.CBH.FST yielded similar results in the TA, but not in the Gastroc. AAV.MHCK7.FST and AAV.CMV.FKRP.pA.MHCK7.FST showed less muscle growth than did CMV vector, while AAV.CMV.FKRP.IRES.FST showed still less growth and AAV.MHCK7.FKRP.IRES.FST showed no growth at all over this time period. No vector expressing only FKRP showed any growth. FST was superior to SMAD7 in inducing muscle growth when MCK was used as the promoter, and as previously shown FST was superior to IGF1 in a similar assay(ns). These data show that rAAV.MHCK7.FKRP.pA.CMV.FST can have a profound effect on muscle growth within two months of treatment, and that use of a two promoter system could yield functional changes that were as good as AAVs that expressed only a single FST gene.

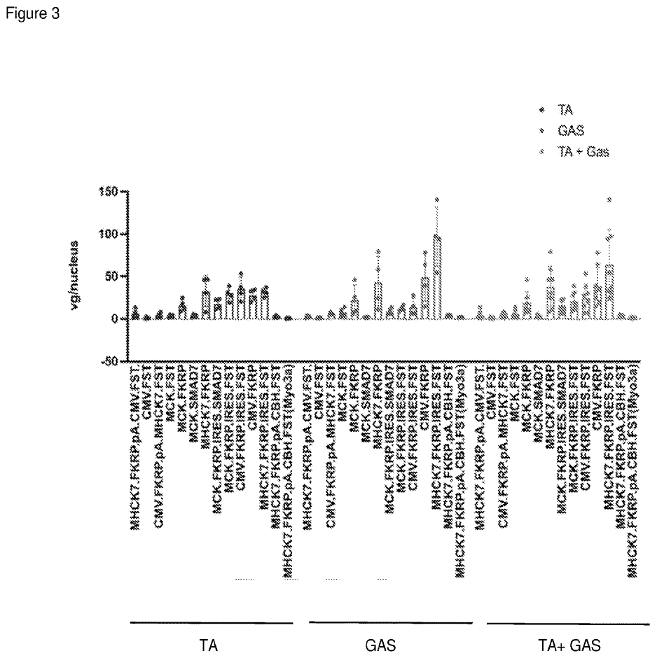

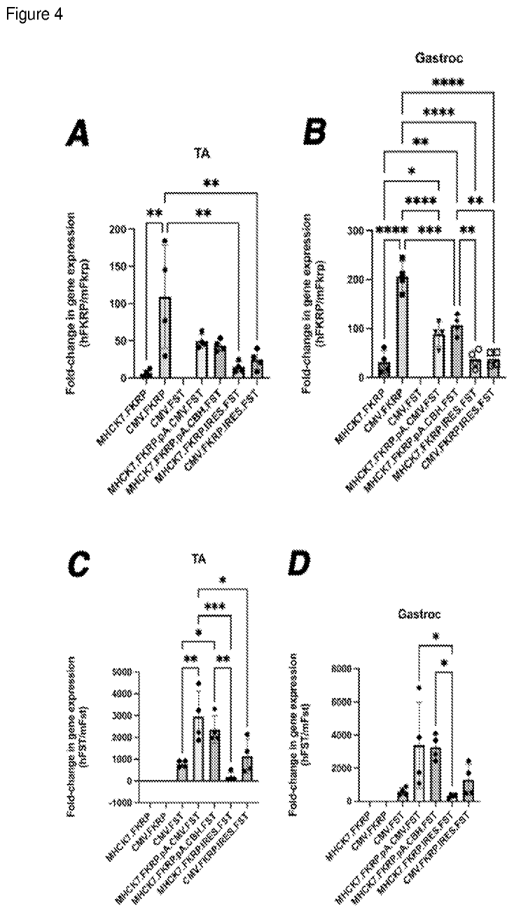

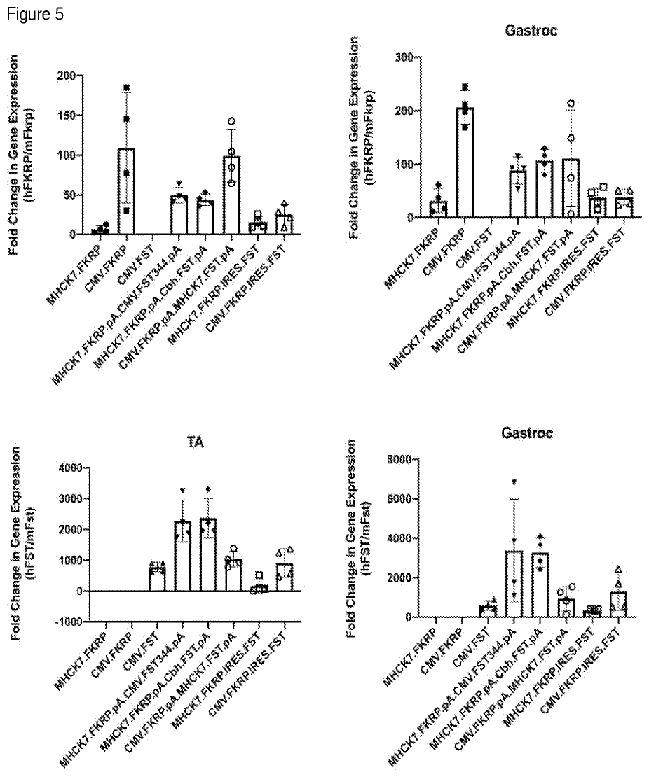

The biodistribution of AAV vector genomes (vg) (FIG. 3) and FKRP and FST gene expression (FIG. 4) was assayed in muscles after IM injection. Bicistronic vectors that delivered the greatest muscle growth actually had the lowest relative levels of muscle transduction. Thus, these AAVs were not more potent simply because there was more gene therapy present. For FKRP and FST gene expression, CMV was a stronger promoter than MHCK7 or MCK. Bicistronic vector utilizing MHCK7 for FKRP gene expression, however, had 3- to 8-fold stronger gene FKRP expression than monocistronic vector using the same MHCK7 promoter in the TA or Gastroc muscle, despite the latter AAV having more vgs present. Similarly, bicistronic vector using CMV for FST gene expression had 3- to 6-fold stronger FST gene expression than monocistronic vector using the same CMV promoter. Here again, vgs in muscle for the two vectors were not significantly different in the TA or Gastroc. Thus, the presence of dual promoters in the bicistronic vector amplified both FKRP and FST expression relative to monocistronic AAVs using the same promoter.

### Example 3

### Muscle Function Changes After Bicistronic or Monocistronic AAV Therapy in FKRPP44L Mice

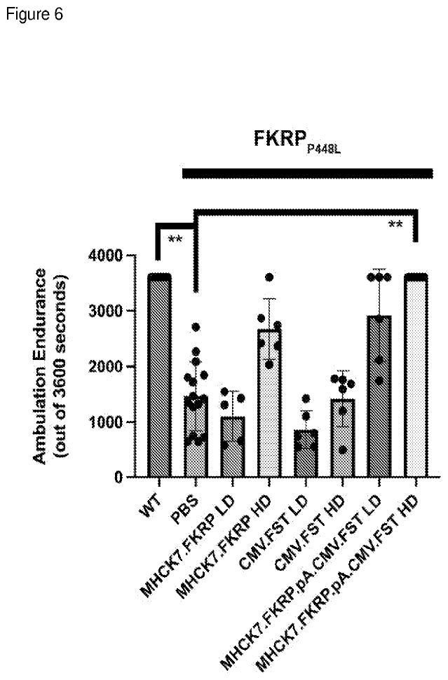

Gene expression for the most potent bicistronic vectors and their monocistronic equivalents after IM injection in wild type muscles was analyzed and the data is provided in FIG. 6. All AAVs are serotype rhesus 74 (rh74). IM injections were done as described in Example 2.

Human FKRP gene expression from the AAV vector, normalized to endogenous mouse Fkrp gene expression, and human FST gene expression, relative to endogenous mouse Fst gene expression, were measured by qRT-PCR, referenced to 18S rRNA expression, in the tibialis anterior (TA) and in the gastrocnemius (Gastroc) muscles. CMV is a stronger promoter than MHCK7. However, use of the two promoter bicistronic vector (either AAV.MHCK7.FKRP.pA.CMV.FST or AAV.MHCK7.FKRP.pA.Cbh.FST) led to greater FKRP gene expression relative to AAV.MHCK7.FKRP alone, and to greater FST gene expression than CMV.FST alone. IRES constructs were inferior to two promoter constructs.

Each promoter contains a unique enhancer element, and both enhancers in such bicistroinic constructs amplified gene expression independent their position relative to the promoters (that is part of the definition of how an enhancer works).

AAV.MHCK7.FKRP.pA.CMV.FST showed stronger gene expression than AAV.MHCK7.FKRP alone for FKRP expression and CMV.FST for FST gene expression. This was also true of Cbh was used in the second position, which also utilizes the same CMV enhancer element. FST gene expression was also stronger in the two promoter bicistronic constructs. Thus, the presence of dual promoters in the bicistronic vector amplified transgene expression for both transgenes.

### Example 4

Muscle Function Changes after Bicistronic or Monocistronic AAV Therapy in FKRP_{P448L} Mice

The AAV.MHCK7.FKRP.pA.CMV.FST vector, and its monocistronic equivalents (AAV.MHCK7.FKRP and AAV.CMV.FST), in the FKRPP_{448L} mouse model of LGMD2I. Mice were administered a high dose (HD, 5×10¹³ vg/kg) or a low dose (LD, 1×10¹³ vg/kg) of these vectors at 1 month of age via intravenous injection into the tail vein (FIG. 6). One week prior to euthanasia at 7 months of age, mice were subjected to 5 days of a 60 minute walk test at 15 m/min. Mice were given three days of practice at this test, then the test was administered once daily for five consecutive days, with all five days averaged to obtain a single ambulation measure. All five days of data were averaged for each data point shown.

On the week of assessment, mice were run on the treadmill for 75 minute tests (5 minutes at 5 meters/minute, with a 1 meter/minute increase over 10 minutes leading to 60 minutes at 15 meters/minute). The mice were run for 5 days prior to euthanasia. Blood was collected pre- and post-ambulation via the sub-mandibular vein. Collected blood was allowed to clot for 1 hour at room temperature, then centrifuged at 1000×g for 10 minutes to separate out the serum. Serum CK was measured using the Creatine Kinase (CK)-SL kit (Sekisui Diagnostics; Burlington, MA) as per manufacturer's protocol.

In our experience, all wild type mice can uniformly walk for an hour in this assay, and this was also the case in this experiment. Untreated mutant (PBS-treated) FKRPP_{448L} mice, by contrast, showed an average decrease in ambulation endurance of 60%. None of the untreated mutant mice showed had a normal level of ambulation endurance. FKRPP_{448L} mice treated with the high dose of AAV.MHCK7.FKRP showed at 50% improvement in ambulation endurance relative to PBS-treated, but only one of six mice showed normal ambulation and this result did not reach significance. The low dose of AAV.MHCK7.FKRP and both dose of AAV.CMV.FST showed no improvement at all in ambulation.

As shown in FIG. 4, LD bicistronic (Bi) AAV.MHCK7.FKRP.pA.CMV.FST showed a 60% improvement in ambulation, on average, relative to Mut, with 3 of 6 mice showing normal ambulation. HD Bi AAV.MCHK7.FKRP.pA.CMV.FST mice showed completely normal ambulation (7 of 7) in all subjects this assay. Thus, one of the most clinically important measures of motor function in muscular dystrophy, ambulation, was completely recovered in all mice with high dose AAV.MHCK7.FKRP.pA.CMV.FST bicistronic vector, a finding that did not occur with either single gene therapy alone at either dose.

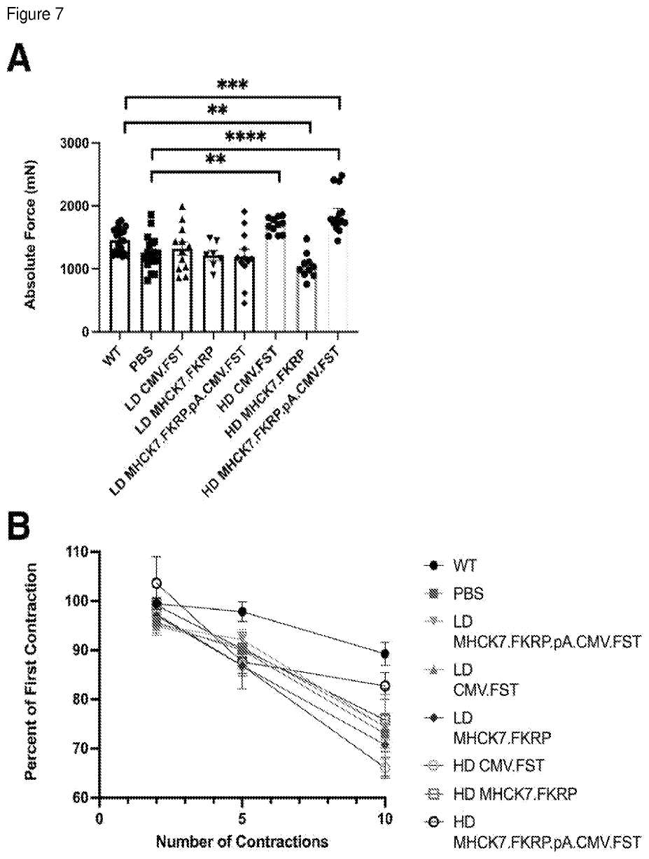

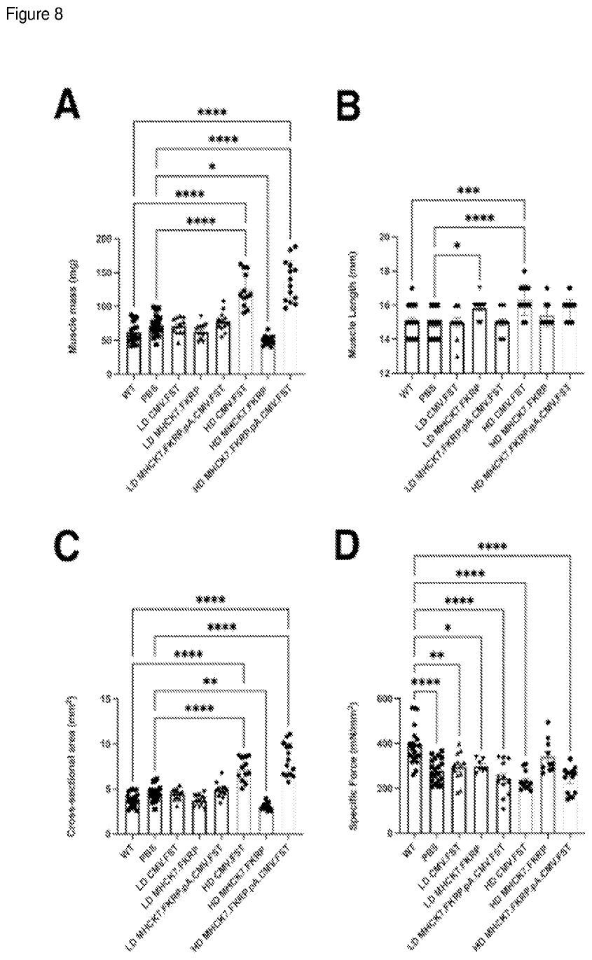

After ambulation studies were completed, maximal tetanic force and force drop during repeated eccentric contractions were measured in situ in the tibialis anterior (TA) muscle (FIGS. 7A, B, FIGS. 8A-D). Mice were anesthetized with ketamine/xylazine, and hindlimb skin was removed to expose the tibialis anterior (TA) muscle. The distal tendon was dissected out and sutured to a force transducer (Aurora Scientific, Aurora, ON). Muscle contractions were produced by stimulation of the sciatic nerve by bipolar platinum electrodes and the optimal length was determined by stretching the muscle until maximum twitch force was elicited. Maximum force was determined by successive stimulation at 50, 100, 150, and 200 Hz with a 1-minute rest period between each stimulation, and specific force was ascertained by dividing the maximum force by the muscle cross-sectional area. Finally, the TA underwent 10 cycles of eccentric contractions, each in which the TA is stimulated for 350 ms total, the last 200 ms of this while being stretched by 10%, before returning to optimum length. For these measurements, the maximum force generated in the first cycle prior to muscle lengthening is designated as 100%.

Absolute force was increased with the high dose of rAAV.CMV.FST or HD rAAV.MHCK7.FKRP.pA.CMV.FST compared to PBS-treated FKRPP448L mice, and rAAV.MHCK7.FKRP.pA.CMV.FST-treated FKRPP448L mice had significantly higher absolute force than WT. For high dose bicistronic FKRP/FST treatment, muscle mass was increased 1.9±0.4-fold and cross-sectional muscle area was increased 1.8±0.4-fold compared to PBS-treated FKRPP448L muscle and was increased 2.2±0.5-fold and 2.1±0.9-fold, respectively, compared to wild type muscle (FIGS. 8A, 8C). Neither low dose rAAV.CMV.FST or LD rAAV.MHCK7.FKRP.pA.CMV.FST significantly changed absolute force, muscle mass, or cross-sectional area, nor did LD rAAV.MHCK7.FKRP. Interestingly, HD rAAV.MHCK7.FKRP muscles were significant smaller than mock-treated FKRP_{P448L}muscles, both in terms of mass and cross-sectional area (FIGS. 8A, 8C). Additionally, high dose AAV.CMV.FST muscles showed significantly increased muscle length relative to mock-treated FKRP_{P448L} and wild type muscles (FIG. 8B).

Both absolute force and specific force (absolute force normalized to muscle cross-sectional area (CSA)) were reduced in untreated FKRP_{P448L} mutant mice at 7 months of age in comparison to age- and gender-matched wild type mice; FKRP_{P448L} mice had a reduction in specific force of 25% and a reduction in absolute force of 20% at either 150 Hz (FIG. 7A, FIG. 7D) or 200 Hz (ns). While HD rAAV.CMV.FST and rAAV.MHCK7.FKRP.pA.CMV.FST therapy increased absolute force significantly (FIG. 8A), this was due to an increase in muscle mass (FIG. 8A). This therefore did not result in a significant increase in specific force (FIG. 8D), which takes relative muscle mass into account.

FKRP_{P448L} mice had a 27±4% force drop by the 10^{th} contraction in an eccentric contraction muscle injury paradigm. While not significant, this was greater than the 11±1% force decrement found in wild type mice (FIG. 7B). rAAV.MHCK7.FKRP.pA.CMV.FST treatment maintained a force deficit near wild type (17±1% vs. 11±1%) at the 10^{th} contraction. By contrast, HD rAAV.CMV.FSTand HD rAAV.MHCK7.FKRP showed force drops of 34±3% and 24±6%, respectively, which was on a par with mock-treated FKRP_{P448L} mice. While this trend suggested some prevention of muscle damage by rAAV.MHCK7.FKRP.pA.CMV.FST during repeated stimulations, this improvement in force drop did not reach significance.

### Example 5

Uneven Induction of Limb Muscle Growth with Single FST Gene Therapy

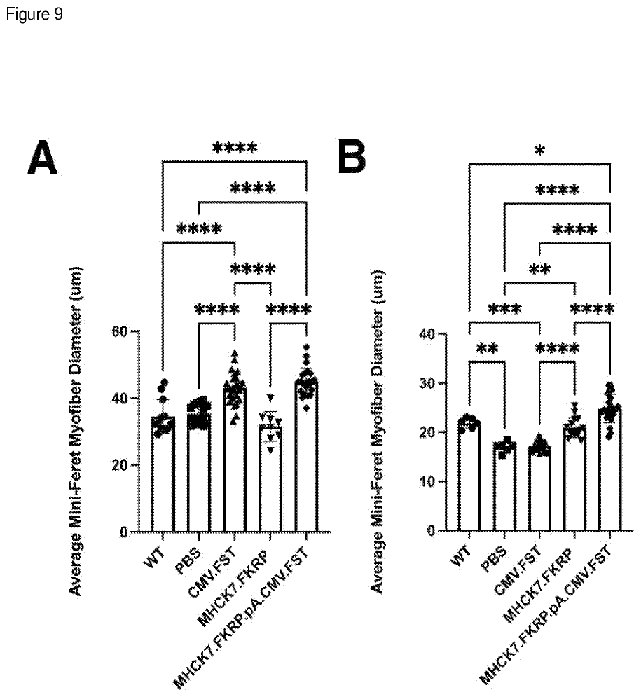

The average Mini-Feret myofiber diameter was quantified for cross-sections of TA and diaphragm muscle (FIG. 9A,B), as well as myofiber number for whole cross-sections of the TA muscle (ns). rAAV.MHCK7.FKRP.pA.CMV.FST therapy increased both the average size of myofibers (+43±4%, FIG. 9A) and myofiber number (by 49±19%) in the TA relative to WT. rAAV.CMV.FST gene therapy showed equivalent increases to bicistronic therapy in the TA, but it did not similarly increase myofiber size in the diaphragm (FIG. 9C); Diaphragm muscle myofiber diameters in FKRP/FST-treated were significantly increased, by 31±2%, relative to FST alone, while FST alone showed no increase relative to wild type or mock-treated FKRP_{P448L} muscles.

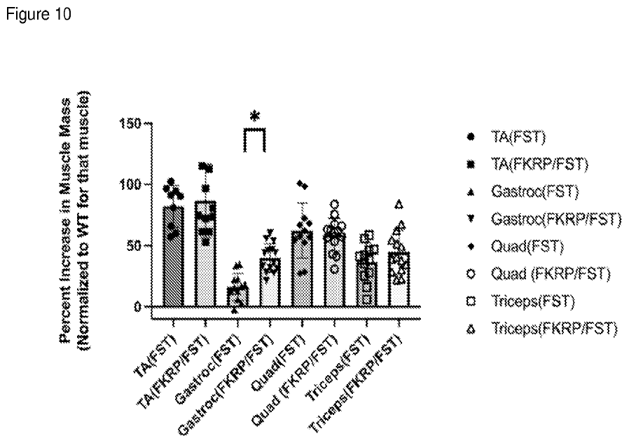

While equivalent percent increase in muscle mass above wild type muscle was observed for rAAV.CMV.FST and rAAV.MHCK7.FKRP.pA.CMV.FST treatments in the TA(+82±17% vs. +87±30%), Quad (+62±22% vs. +59±14%), and Triceps (+37±16% vs. +45±18%) muscles, there was a significantly difference in muscle growth for the Gastroc (+16±11% for FST vs. +40±12% for FKRP/FST) (FIG. 10). Thus, induction of growth amongst individual muscles was more even with FKRP/FST bicistronic therapy than it was for FST therapy alone, with large differences seen in the gastrocnemius and the diaphragm.

### Example 6

### Comparison of Bicistronic Vector and Monocistronic Biodistribution and AAV-Induced Gene Expression

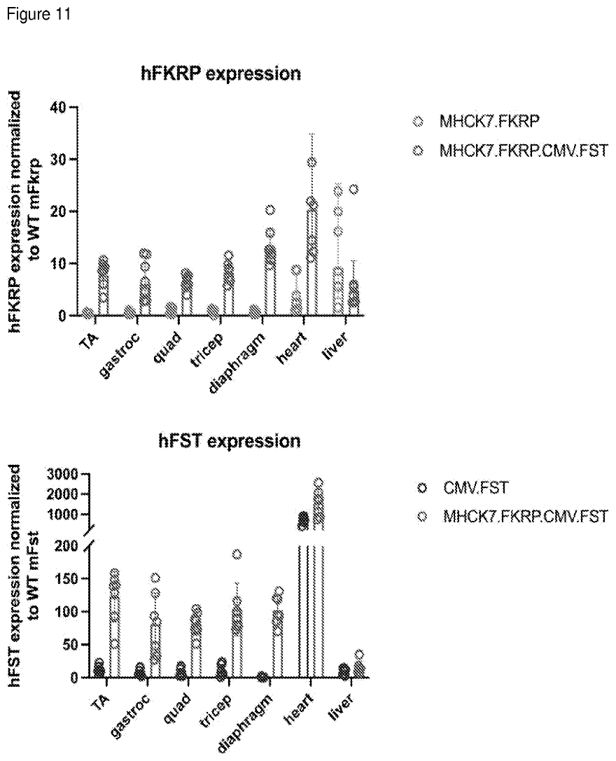

Gene expression in the FKRP_{P448L} mice treated IV with the various AAV vectors was also measured using skeletal muscles (tibialis anterior, gastrocnemius, quadriceps femoris, triceps brachii, and diaphragm), heart and liver taken from high dose (5×10¹³ vg/kg) mice. Human FKRP gene expression from the AAV vector, normalized to wild type mouse FKRP gene expression, and human FST gene expression, relative to endogenous wild type mouse FST gene expression, were measured by qRT-PCR, referenced to 18S rRNA expression. As shown in FIG. 11, an even greater amplification of human FKRP and human FST gene expression with the AAV.MHCK7.FKRP.pA.CMV.FST vector compared to either AAV.MHCK7.FKRP or AAV.CMV.FST alone. The high dose allows for greater than normal amounts of therapeutic gene expression in skeletal muscles throughout the body plan. Thus, the effects of amplification of therapeutic gene expression were greater in the FKRP_{P448L} mouse than they were in wild type mice. These data clearly show the profound benefit of having two enhancers that can act synergistically to amplify dual transgene expression in skeletal muscles that have muscular dystrophy. Such a therapy can recover lost walking ability, essentially reversing disease, something single gene replacement therapy has not been shown to do. In addition, this bicistronic formulation amplifies the level of expression of both therapeutic genes relative to the use of single promoters alone.

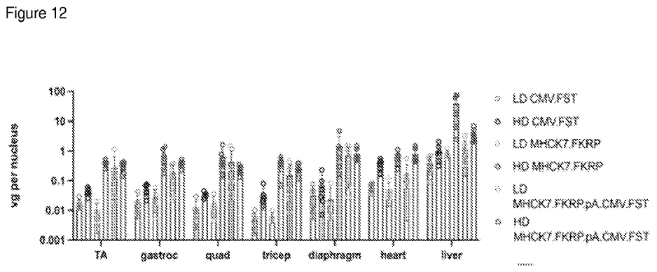

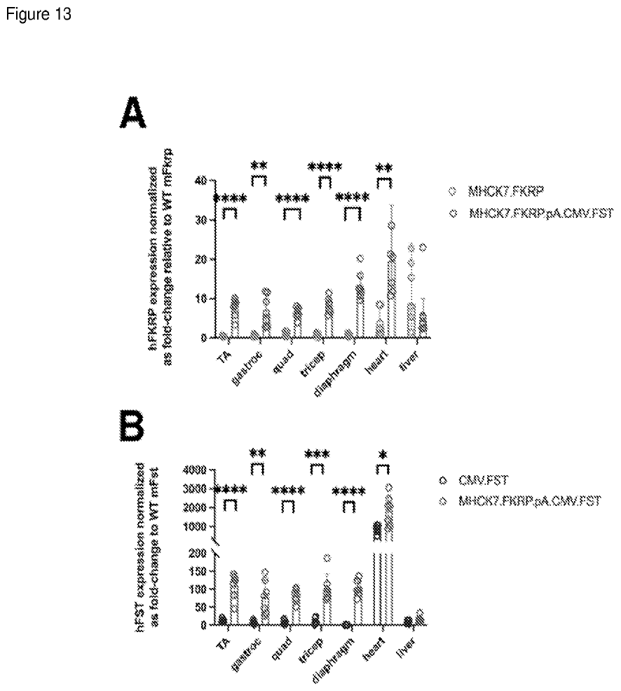

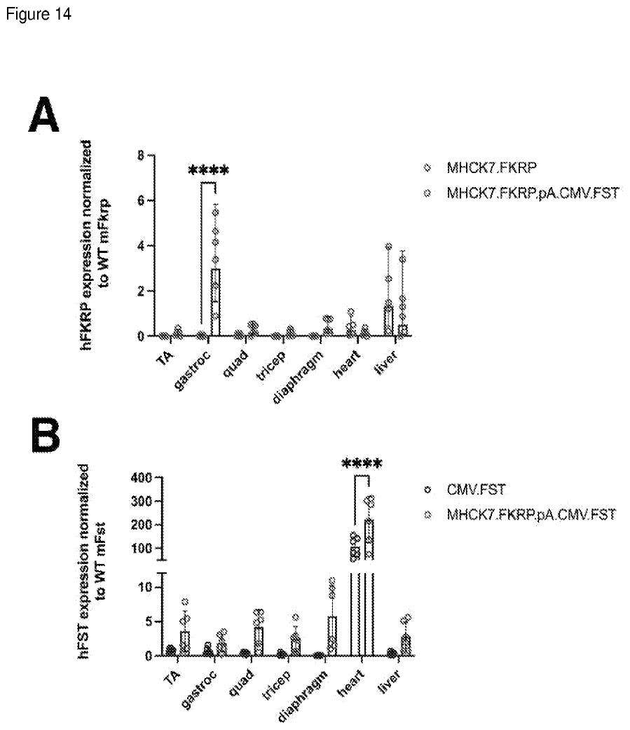

In addition, AAV biodistribution (FIG. 12) and FKRP and FST gene expression (FIG. 13 and FIG. 14) was measured in the FKRP_{P448L} mice treated with the various AAV vectors. Certain limb muscles (TA, gastric, quad, triceps), diaphragm, heart and liver were analyzed. There were several findings of interest: First, rAAV.CMV.FST therapy showed about 10-fold lower overall transduction in skeletal muscles compared to rAAV.MHCK7.FKRP or MHCK7.FKRP.pA.CMV.FST, while vg levels for the three AAVs were not significantly different in heart or liver (FIG. 12). FKRP was known to prevent skeletal muscle damage in FKRP_{P448L} mice, which would lead to prolonged muscle transduction, while FST cannot, thereby causing elimination of transduced myofibers. Vg levels for rAAV.MHCK7.FKRP and rAAV.MHCK7.FKRP.pA.CMV.FST were similar in all tissues studied, with single FKRP vector showing slightly higher levels in all instances. At the high dose, an amplification of FKRP and FST gene expression was observed with rAAV.MHCK7.FKRP.pA.CMV.FST compared to either rAAV.MHCK7.FKRP or rAAV.CMV.FST alone (FIG. 7). Low dose results were more variable (FIG. 13). These data suggest that the presence of two enhancers within the two individual promoter elements can act synergistically to amplify expression for both transgenes, both in FKRPP448L muscles (FIG. 7) and in wild type muscles (FIG. 4).

### Example 7

### Induction of FKRP and FST Protein Expression in Muscle, Liver and Serum

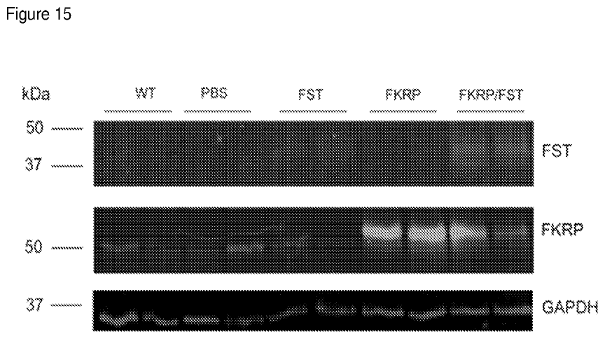

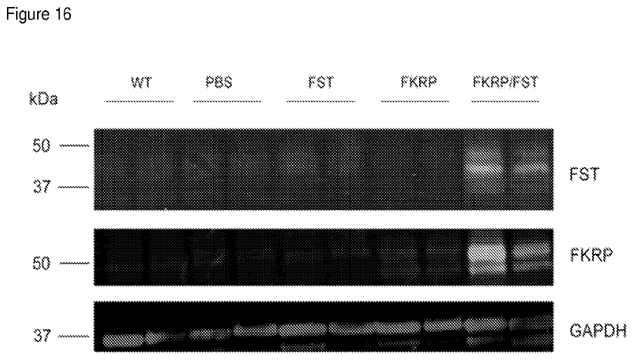

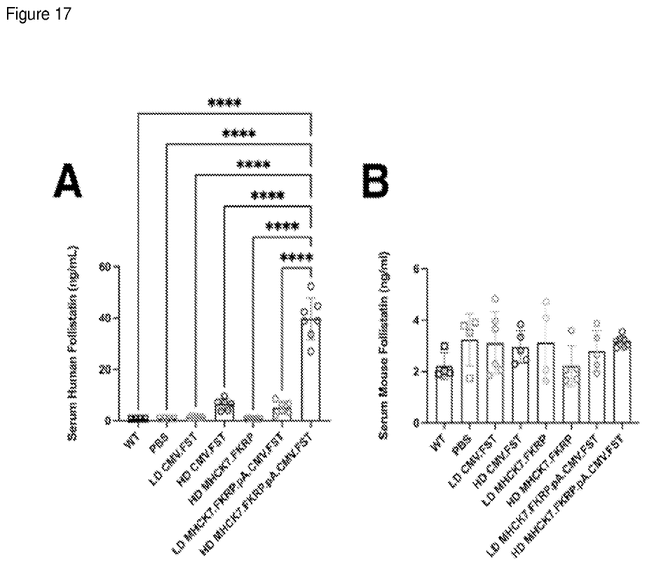

An antiserum against FKRP protein was used to identify increased FKRP protein expression in both rAAV.MHCK7.FKRP and rAAV.MHCK7.FKRP.pA.CMV.FST-treated liver and muscle tissue (FIGS. 15, 16). Differences in FST protein expression were more difficult to observe by western blot, but FST protein appeared elevated with HD bicistronic treatment in both liver and muscle (FIGS. 15, 16). FST is a secreted protein that can have affects in trans after its secretion into serum. Therefore, ELISA assays were performed to measure serum levels of human FST protein (FIG. 17A), which would derive from the delivered gene therapy, and endogenous mouse FST protein (FIG. 17B). Endogenous mouse FST protein averaged 2.2±0.5 ng/mL in wild type mouse serum and 3.2±1 ng/mL in PBS-treated FKRP_{P448L} mouse serum. Serum levels of mouse FST were maintained in this range for all other treatment groups. Serum human FST protein was elevated to 6.4±2.2 ng/mL in HD rAAV.CMV.FST, 5.1±2.5 ng/mL in LD rAAV.MHCK7.FKRP.pA.CMV.FST, and 39.7±8 ng/mL in HD rAAV.MHCK7.FKRP.pA.CMV.FST (a 12±3-fold increase over endogenous mouse FST).

### Example 8

### Induction of AAV-Induced aDG Glycosylation

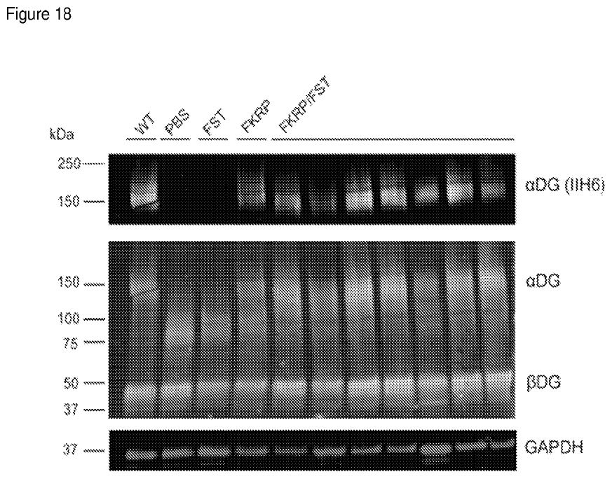

Immunoblotting with IIH6, an antibody that recognizes functional glycosylation of aDG (Ervasti et al. J Cell Biol 122: 809-823, 1993), and with an anti-peptide polyclonal antibody that recognizes aDG and J3DG protein (FIG. 18). 11H6 will identify matriglycan expression, which is required for laminin binding to aDG (Goddeeris et al., Nature 503: 136-140, 2013), and while the anti-peptide polyclonal antiserum will detect total protein expression for both aDG and J3DG. WT muscle showed a strong IIH6 immunoblot signal at 156 kDa, the native molecular weight of functionally glycosylated aDG in skeletal muscle (Ervasti et al., Cell 66: 1121-1131, 1991), while PBS-treated and FST-treated FKRP_{P448L} muscles showed no IIH6 signal and a reduced molecular weight for the aDG polypeptide consistent with reduced glycosylation (Michele et al., Nature 418: 417-422, 2002). Both rAAV.MHCK7.FKRP and multiple rAAV.MHCK7.FKRP.pA.CMV.FST muscle samples showed increased expression of IIH6 with a protein molecular weight of 156 kDa. The 43 kDa band for bDG protein was present in all samples, as expected based on previous studies (Brockington et al., Am J Hum Genet 69: 1198-1209, 2001, Brown et al., Am J Pathol 164: 727-737, 2004).

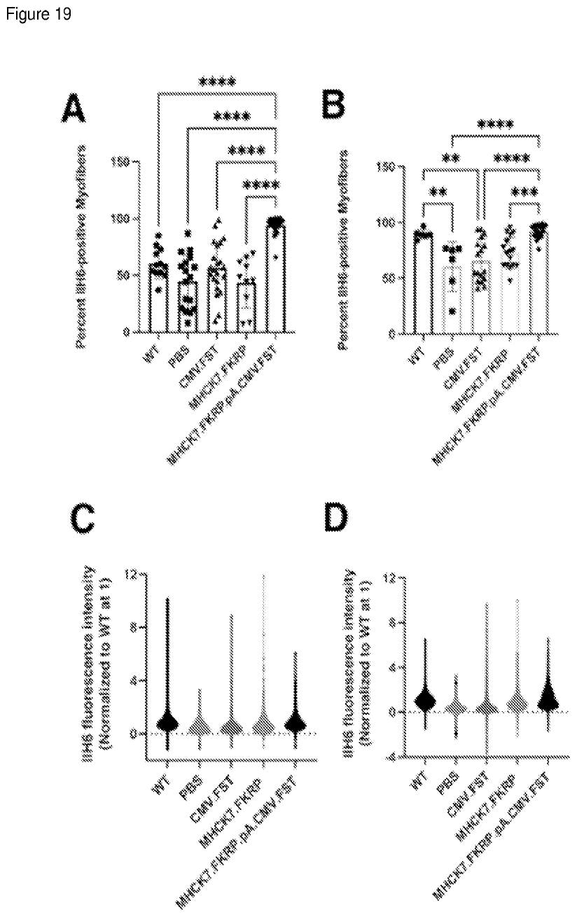

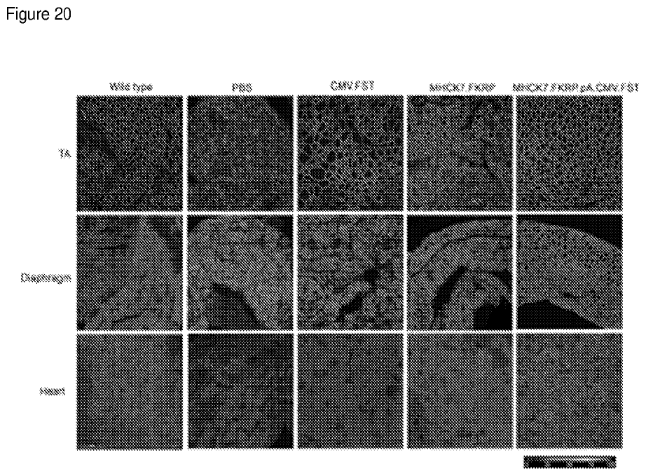

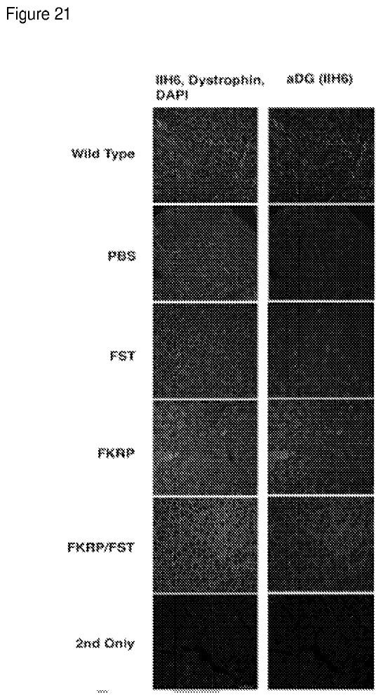

Quantification of IIH6 immunostaining was also done to assess expression of functional aDG glycosylation in TA, diaphragm, and heart (FIGS. 19-22). Dystrophin and DAPI co-staining were done to identify myofiber membranes and nuclei, respectively. The percentage of myofibers with at least 50% coincident IIH6 membrane staining was significantly reduced in PBS-treated FKRP_{P448L} muscles compared to WT for both the TA and diaphragm muscle, and this was significantly increased in rAAV.MHCK7.FKRP.pA.CMV.FST-treated muscles in both instances. Because the P448L mutation is a partial loss of function mutation (Chan et al. Hum Mol Genet 19: 3995-4006, 2010), and because regenerating muscles that arise after myofiber damage typically have greater IIH6 expression (Cohn et al., Cell 110: 639-648, 2002; Krag et al. Skelet Muscle 1: 31, 2011), there were still positively stained myofibers in FKRP_{P448L} muscles. Both the percentage of positively stained myofibers and the intensity of IIH6 staining were significantly increased in rAAV.MHCK7-FKRP.pA.CMV.FST-treated FKRPP448L TA and diaphragm to near or above WT levels (FIG. 19). The percentage of the membrane that showed continuous IIH6 membrane staining was dramatically increased in rAAV.MHCK7-FKRP.pA.CMV.FST-treated FKRPP448L muscles compared to all other conditions (FIG. 20). These data suggest that functional aDG glycosylation is not only expressed along more myofibers after bicistronic FKRP/FST gene therapy, but that this glycosylation is stronger per unit membrane area.

### Example 9

### Effects on Muscle Pathology

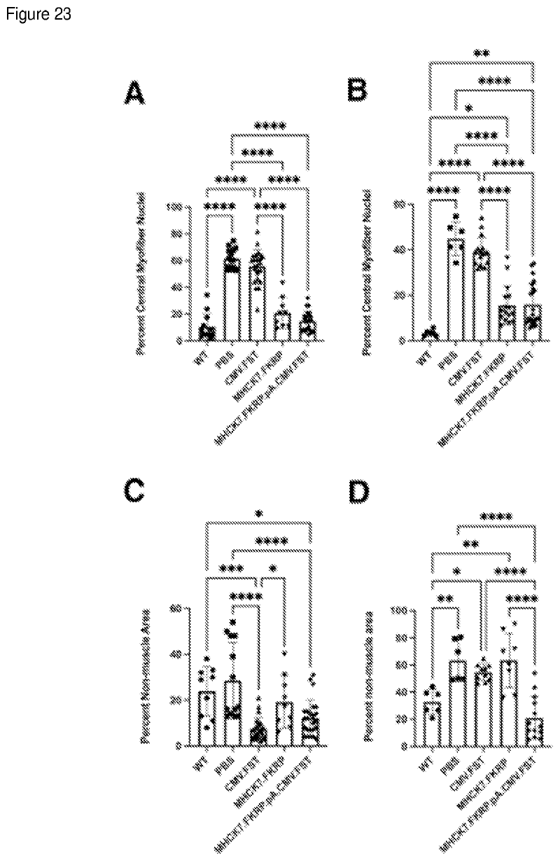

Myofibers that become damaged or destroyed in mice with muscular dystrophy can regenerate to make newly formed myofibers. When such regeneration occurs, nuclei typically remain within the center of newly formed skeletal myofibers, and this provides an indelible marker of such a cycles of degeneration and regeneration. Both TA and diaphragm muscles of mock (PBS)-treated FKRP_{P448L} mice showed a dramatic increase in the percentage of myofibers with centrally located nuclei compared to age- and gender-matched wild type mice (FIG. 23A,B). rAAV.CMV.FST-treated FKRPP448L muscles also had highly elevated myofibers with central nuclei, while rAAV.MHCK.FKRP and rAAV.MHCK7.FKRP.pA.CMV.FST treatment significantly reduced this level in the TA and the diaphragm.

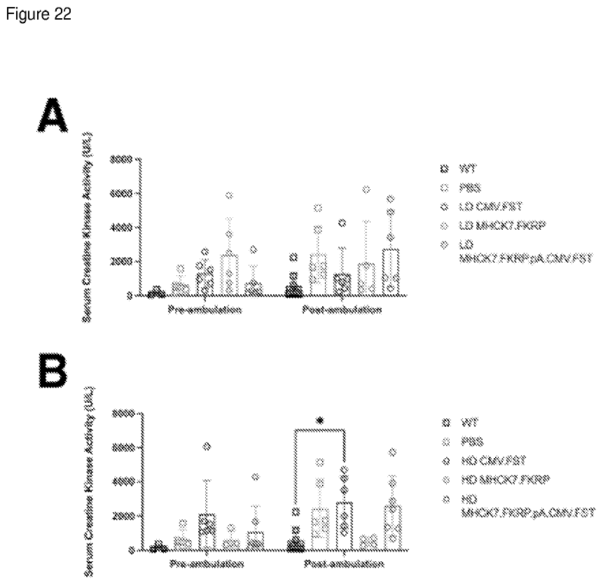

Release of creatine kinase into the serum is another measure of muscle damage in dystrophic mice. Serum CK activity levels were elevated in FKRP_{P448L} mice relative to wild type both before and after their five day ambulation protocol, and elevations for both WT and FKRPP448L were larger post-ambulation (FIG. 22). Both low dose (LD) and high dose (HD) CMV.FST showed no improvement in serum CK. HD rAAV.MHCK7.FKRP and HD rAAV.MHCK7.FKRP.pA.CMV.FST showed trends toward lower serum CK levels in the pre-ambulation group with HD but not LD. This trend toward lower serum CK levels for HD FKRP/FST therapy was lost in the post-ambulation group.

We next measured the percentage of non-muscle area within muscles (FIG. 23). This is a measure of muscle wasting, where non-muscle tissue replaces muscle cells as muscular dystrophy progresses in severity. Percentage non-muscle area was increased in PBS-treated FKRP_{P448L} mice relative to wild type (WT) in both the TA (FIG. 23C) and diaphragm (FIG. 22D). For both the TA and diaphragm, the percentage non-muscle area showed an inverted pattern relative to the induction of muscle growth (FIG. 9). The percentage non-muscle area was decreased in the TA by both rAAV.CMV.FST and rAAV.MHCK7.FKRP.pA.CMV.FST, both of which induced muscle growth in that muscle, while for diaphragm, percentage non-muscle area was only reduced to normal levels by FKRP/FST bicistronic therapy. This again was consistent with the fact that only bicistronic therapy induced muscle growth in diaphragm.

Sequences
pAAV.MHCK7.FKRP.spA.CMV.spA KanR
(SEQ ID NO: 1)
GTTGGGTGCACGAGTGGGTTACATCGAACTGGATCTCAACAGCGGTAAGATCCTTGAGAGTTTTCGCC
CCGAAGAACGAATTCTTAGAAAAACTCATCGAGCATCAAATGAAACTGCAATTTATTCATATCAGGAT
TATCAATACCATATTTTTGAAAAAGCCGTTTCTGTAATGAAGGAGAAAACTCACCGAGGCAGTTCCAT
AGGATGGCAAGATCCTGGTATCGGTCTGCGATTCCGACTCGTCCAACATCAATACAACCTATTAATTT
CCCCTCGTCAAAAATAAGGTTATCAAGTGAGAAATCACCATGAGTGACGACTGAATCCGGTGAGAATG
GCAAAAGTTTATGCATTTCTTTCCAGACTTGTTCAACAGGCCAGCCATTACGCTCGTCATCAAAATCA
CTCGCATCAACCAAACCGTTATTCATTCGTGATTGCGCCTGAGCGAGACGAAATACGCGATCGCTGTT
AAAAGGACAATTACAAACAGGAATCGAATGCAACCGGCGCAGGAACACTGCCAGCGCATCAACAATAT
TTTCACCTGAATCAGGATATTCTTCTAATACCTGGAATGCTGTTTTCCCGGGGATCGCAGTGGTGAGT
AACCATGCATCATCAGGAGTACGGATAAAATGCTTGATGGTCGGAAGAGGCATAAATTCCGTCAGCCA
GTTTAGTCTGACCATCTCATCTGTAACATCATTGGCAACGCTACCTTTGCCATGTTTCAGAAACAACT
CTGGCGCATCGGGCTTCCCATACAATCGATAGATTGTCGCACCTGATTGCCCGACATTATCGCGAGCC
CATTTATACCCATATAAATCAGCATCCATGTTGGAATTTAATCGCGGCCTAGAGCAAGACGTTTCCCG
TTGAATATGGCTCATAACACCCCTTGTATTACTGTTTATGTAAGCAGACAGTTTTATTGTTCATGACC
AAAATCCCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTGTCTTACCGGGT
TGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGTTATCCCCTGATTCTGTGG
ATAACCGTATTACCGCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCGCGTTGGCCGATTCA
TTAATGCAGCTGCGCGCTCGCTCGCTCACTGAGGCCGCCCGGGCAAAGCCCGGGCGTCGGGCGACCTT
TGGTCGCCCGGCCTCAGTGAGCGAGCGAGCGCGCAGAGAGGGAGTGGCCAACTCCATCACTAGGGGTT
CCTTGTAGTTAATGATTAACCCGCCATGCTACTTATCTACGTAGCCATGCTCTAGAGTTTAAACAAGC
TCTAGAGTTTAAACAAGCTTGCATGTCTAAGCTAGACCCTTCAGATTAAAAATAACTGAGGTAAGGGC
CTGGGTAGGGGAGGTGGTGTGAGACGCTCCTGTCTCTCCTCTATCTGCCCATCGGCCCTTTGGGGAGG
AGGAATGTGCCCAAGGACTAAAAAAAGGCCATGGAGCCAGAGGGGCGAGGGCAACAGACCTTTCATGG
GCAAACCTTGGGGCCCTGCTGTCTAGCATGCCCCACTACGGGTCTAGGCTGCCCATGTAAGGAGGCAA
GGCCTGGGGACACCCGAGATGCCTGGTTATAATTAACCCAGACATGTGGCTGCCCCCCCCCCCCCAAC
ACCTGCTGCCTCTAAAAATAACCCTGTCCCTGGTGGATCCCCTGCATGCGAAGATCTTCGAACAAGGC
TGTGGGGGACTGAGGGCAGGCTGTAACAGGCTTGGGGGCCAGGGCTTATACGTGCCTGGGACTCCCAA
AGTATTACTGTTCCATGTTCCCGGCGAAGGGCCAGCTGTCCCCCGCCAGCTAGACTCAGCACTTAGTT
TAGGAACCAGTGAGCAAGTCAGCCCTTGGGGCAGCCCATACAAGGCCATGGGGCTGGGCAAGCTGCAC
GCCTGGGTCCGGGGTGGGCACGGTGCCCGGGCAACGAGCTGAAAGCTCATCTGCTCTCAGGGGCCCCT
CCCTGGGGACAGCCCCTCCTGGCTAGTCACACCCTGTAGGCTCCTCTATATAACCCAGGGGCACAGGG
GCTGCCCTCATTCTACCACCACCTCCACAGCACAGACAGACACTCAGGAGCAGCCAGCGGCGCGCCCA
GGTAAGTTTAGTCTTTTTGTCTTTTATTTCAGGTCCCGGATCCGGTGGTGGTGCAAATCAAAGAACTG
CTCCTCAGTGGATGTTGCCTTTACTTCTAGGCCTGTACGGAAGTGTTACTTCTGCTCTAAAAGCTGCG
GAATTGTACCCGCGGCCGGCTAGCCGCCACCATGCGGCTCACCCGCTGCCAGGCTGCCCTGGCGGCCG
CCATCACCCTCAACCTTCTGGTCCTCTTCTATGTCTCGTGGCTGCAGCACCAGCCTAGGAATTCCCGG
GCCCGGGGGCCCCGTCGTGCCTCTGCTGCCGGCCCCCGTGTCACCGTCCTGGTGCGGGAGTTCGAGGC
ATTTGACAACGCGGTGCCCGAGCTGGTAGACTCCTTCCTGCAGCAAGACCCAGCCCAGCCCGTGGTGG
TGGCAGCCGACACGCTCCCCTACCCGCCCCTGGCCCTGCCCCGCATCCCCAACGTGCGTCTGGCGCTG
CTCCAGCCCGCCCTGGACCGGCCAGCCGCAGCCTCGCGCCCGGAGACCTACGTGGCCACCGAGTTTGT
GGCCCTAGTACCTGATGGGGCGCGGGCTGAGGCACCTGGCCTGCTGGAGCGCATGGTGGAGGCGCTCC
GCGCAGGAAGCGCACGTCTGGTGGCCGCCCCGGTTGCCACGGCCAACCCTGCCAGGTGCCTGGCCCTG
AACGTCAGCCTGCGAGAGTGGACCGCCCGCTATGGCGCAGCCCCCGCCGCGCCCCGCTGCGACGCCCT
GGACGGAGATGCTGTGGTGCTCCTGCGCGCCCGCGACCTCTTCAACCTCTCGGCGCCCCTGGCCCGGC
CGGTGGGCACCAGCCTCTTTCTGCAGACCGCCCTTCGCGGCTGGGCGGTGCAGCTGCTGGACTTGACC
TTCGCCGCGGCGCGCCAGCCCCCGCTGGCCACGGCCCACGCGCGCTGGAAGGCTGAGCGCGAGGGACG
CGCTCGGCGGGCGGCGCTGCTCCGCGCGCTGGGCATCCGCCTAGTGAGCTGGGAAGGCGGGCGGCTGG
AGTGGTTCGGCTGCAACAAGGAGACCACGCGCTGCTTCGGAACCGTGGTGGGCGACACGCCCGCCTAC
CTCTACGAGGAGCGCTGGACGCCCCCCTGCTGCCTGCGCGCGCTGCGCGAGACCGCCCGCTATGTGGT
GGGCGTGCTGGAGGCTGCGGGCGTGCGCTACTGGCTCGAGGGCGGCTCACTGCTGGGGGCCGCCCGCC
ACGGGGACATCATCCCATGGGACTACGACGTGGACCTGGGCATCTACTTGGAGGACGTGGGCAACTGC
GAGCAGCTGCGGGGGGCAGAGGCCGGCTCGGTGGTGGATGAGCGCGGCTTCGTATGGGAGAAGGCGGT
CGAGGGCGACTTTTTCCGCGTGCAGTACAGCGAAAGCAACCACTTGCACGTGGACCTGTGGCCCTTCT
ACCCCCGCAATGGCGTCATGACCAAGGACACGTGGCTGGACCACCGGCAGGATGTGGAGTTTCCCGAG
CACTTCCTGCAGCCGCTGGTGCCCCTGCCCTTTGCCGGCTTCGTGGCGCAGGCGCCTAACAACTACCG
CCGCTTCCTGGAGCTCAAGTTCGGGCCCGGGGTCATCGAGAACCCCCAGTACCCCAACCCGGCACTGC
TGAGTCTGACGGGAAGCGGCTGAGCATGCACTAGTGCGGCCGCAATAAAAGATCTTTATTTTCATTAG
ATCTGTGTGTTGGTTTTTTGTGTGTCTAGAGCTTCGTTACATAACTTACGGTAAATGGCCCGCCTGGC
TGACCGCCCAACGACCCCCGCCCATTGACGTCAATAATGACGTATGTTCCCATAGTAACGCCAATAGG
GACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAAC
AACTCCGCCCCATTGACGCAAATGGGCGGTAGGCGTGTACGGTGGGAGGTCTATATAAGCAGAGCTCG
TTTAGTGAACCGTCAGATCGCCTGGAGACGCCATCCACGCTGTTTTGACCTCCATAGAAGACACCGGG
ACCGATCCAGCCTCCGGACTCTAGAGGATCCGGTACTCGAGGAACTGAAAAACCAGAAAGTTAACTGG
TAAGTTTAGTCTTTTTGTCTTTTATTTCAGGTCCCGGATCCGGTGGTGGTGCAAATCAAAGAACTGCT
CCTCAGTGGATGTTGCCTTTACTTCTAGGCCTGTACGGAAGTGTTACTTCTGCTCTAAAAGCTGCGGA
ATTGTACCCGCGGCCGCACCATGGTCCGCGCGAGGCACCAGCCGGGTGGGCTTTGCCTCCTGCTGCTG
CTGCTCTGCCAGTTCATGGAGGACCGCAGTGCCCAGGCTGGGAACTGCTGGCTCCGTCAAGCGAAGAA
CGGCCGCTGCCAGGTCCTGTACAAGACCGAACTGAGCAAGGAGGAGTGCTGCAGCACCGGCCGGCTGA
GCACCTCGTGGACCGAGGAGGACGTGAATGACAACACACTCTTCAAGTGGATGATTTTCAACGGGGGC
GCCCCCAACTGCATCCCCTGTAAAGAAACGTGTGAGAACGTGGACTGTGGACCTGGGAAAAAATGCCG
AATGAACAAGAAGAACAAACCCCGCTGCGTCTGCGCCCCGGATTGTTCCAACATCACCTGGAAGGGTC
CAGTCTGCGGGCTGGATGGGAAAACCTACCGCAATGAATGTGCACTCCTAAAGGCAAGATGTAAAGAG
CAGCCAGAACTGGAAGTCCAGTACCAAGGCAGATGTAAAAAGACTTGTCGGGATGTTTTCTGTCCAGG
CAGCTCCACATGTGTGGTGGACCAGACCAATAATGCCTACTGTGTGACCTGTAATCGGATTTGCCCAG
AGCCTGCTTCCTCTGAGCAATATCTCTGTGGGAATGATGGAGTCACCTACTCCAGTGCCTGCCACCTG
AGAAAGGCTACCTGCCTGCTGGGCAGATCTATTGGATTAGCCTATGAGGGAAAGTGTATCAAAGCAAA
GTCCTGTGAAGATATCCAGTGCACTGGTGGGAAAAAATGTTTATGGGATTTCAAGGTTGGGAGAGGCC
GGTGTTCCCTCTGTGATGAGCTGTGCCCTGACAGTAAGTCGGATGAGCCTGTCTGTGCCAGTGACAAT
GCCACTTATGCCAGCGAGTGTGCCATGAAGGAAGCTGCCTGCTCCTCAGGTGTGCTACTGGAAGTAAA
GCACTCCGGATCTTGCAACTCCATTTCGGAAGACACCGAGGAAGAGGAGGAAGATGAAGACCAGGACT
ACAGCTTTCCTATATCTTCTATTCTAGAGTGGTAAACTAGTGCGGCCGCAATAAAAGATCTTTATTTT
CATTAGATCTGTGTGTTGGTTTTTTGTGTGTCTAGAGCATGGCTACGTAGATAAGTAGCATGGCGGGT
TAATCATTAACTACAAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAGCGAGCG
AGCGCGCAGCTGGCGTAATAGCGAAGAGGCCCGCACCGATCGCCCTTCCCAACAGTTGCGCAGCCTGA
ATGGCGAATGGCGATTCCGTTGCAATGGCTGGCGGTAATATTGTTCTGGATATTACCAGCAAGGCCGA
TAGTTTGAGTTCTTCTACTCAGGCAAGTGATGTTATTACTAATCAAAGAAGTATTGCGACAACGGTTA
ATTTGCGTGATGGACAGACTCTTTTACTCGGTGGCCTCACTGATTATAAAAACACTTCTCAGGATTCT
GGCGTACCGTTCCTGTCTAAAATCCCTTTAATCGGCCTCCTGTTTAGCTCCCGCTCTGATTCTAACGA
GGAAAGCACGTTATACGTGCTCGTCAAAGCAACCATAGTACGCGCCCTGTAGCGGCGCATTAAGCGCG
GCGGGTGTGGTGGTTACGCGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTCACGT
AGTGGGCCATCGCCCTGATAGACGGTTTTTCGCCCTTTGACGTTGGAGTCCACGTTCTTTAATAGTGG
ACTCTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAACAAA
ATATTAACGCTTACAATTTAAATATTTGCTTATACAATCTTCCTGTTTTTGGGGCTTTTCTGATTATC
AACCGGGGTACATATGATTGACATGCTAGTTTTACGATTACCGTTCATCGATTCTCTTGTTTGCTCCA
GACTCTCAGGCAATGACCTGATAGCCTTTGTAGAGACCTCTCAAAAATAGCTACCCTCTCCGGCATGA
ATTTATCAGCTAGAACGGTTGAATATCATATTGATGGTGATTTGACTGTCTCCGGCCTTTCTCACCCG
TTTGAATCTTTACCTACACATTACTCAGGCATTGCATTTAAAATATATGAGGGTTCTAAAAATTTTTA
TCCTTGCGTTGAAATAAAGGCTTCTCCCGCAAAAGTATTACAGGGTCATAATGTTTTTGGTACAACCG
ATTTAGCTTTATGCTCTGAGGCTTTATTGCTTAATTTTGCTAATTCTTTGCCTTGCCTGTATGATTTA
TTGGATGTTGGAATCGCCTGATGCGGTATTTTCTCCTTACGCATCTGTGCGGTATTTCACACCGCATA
TGGTGCACTCTCAGTACAATCTGCTCTGATGCCGCATAGTTAAGCCAGCCCCGACACCCGCCAACACC
CGCTGACGCGCCCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGACGAAAGGGCCTCGTGATA
CGCCTATTTTTATAGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATCCGCTCATGAGAC
AATAACCCTGATAAATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACGCTGGTGAAAGT
AAAAGATGCTGAAGATCA
FKRP DNA sequence
SEQ ID NO: 2
ATGCGGCTCACCCGCTGCCAGGCTGCCCTGGCGGCCGCCATCACCCTCAACCTTCTGGTCCTCTTCTA
TGTCTCGTGGCTGCAGCACCAGCCTAGGAATTCCCGGGCCCGGGGGCCCCGTCGTGCCTCTGCTGCCG
GCCCCCGTGTCACCGTCCTGGTGCGGGAGTTCGAGGCATTTGACAACGCGGTGCCCGAGCTGGTAGAC
TCCTTCCTGCAGCAAGACCCAGCCCAGCCCGTGGTGGTGGCAGCCGACACGCTCCCCTACCCGCCCCT
GGCCCTGCCCCGCATCCCCAACGTGCGTCTGGCGCTGCTCCAGCCCGCCCTGGACCGGCCAGCCGCAG
CCTCGCGCCCGGAGACCTACGTGGCCACCGAGTTTGTGGCCCTAGTACCTGATGGGGCGCGGGCTGAG
GCACCTGGCCTGCTGGAGCGCATGGTGGAGGCGCTCCGCGCAGGAAGCGCACGTCTGGTGGCCGCCCC
GGTTGCCACGGCCAACCCTGCCAGGTGCCTGGCCCTGAACGTCAGCCTGCGAGAGTGGACCGCCCGCT
ATGGCGCAGCCCCCGCCGCGCCCCGCTGCGACGCCCTGGACGGAGATGCTGTGGTGCTCCTGCGCGCC
CGCGACCTCTTCAACCTCTCGGCGCCCCTGGCCCGGCCGGTGGGCACCAGCCTCTTTCTGCAGACCGC
CCTTCGCGGCTGGGCGGTGCAGCTGCTGGACTTGACCTTCGCCGCGGCGCGCCAGCCCCCGCTGGCCA
CGGCCCACGCGCGCTGGAAGGCTGAGCGCGAGGGACGCGCTCGGCGGGCGGCGCTGCTCCGCGCGCTG
GGCATCCGCCTAGTGAGCTGGGAAGGCGGGCGGCTGGAGTGGTTCGGCTGCAACAAGGAGACCACGCG
CTGCTTCGGAACCGTGGTGGGCGACACGCCCGCCTACCTCTACGAGGAGCGCTGGACGCCCCCCTGCT
GCCTGCGCGCGCTGCGCGAGACCGCCCGCTATGTGGTGGGCGTGCTGGAGGCTGCGGGCGTGCGCTAC
TGGCTCGAGGGCGGCTCACTGCTGGGGGCCGCCCGCCACGGGGACATCATCCCATGGGACTACGACGT
GGACCTGGGCATCTACTTGGAGGACGTGGGCAACTGCGAGCAGCTGCGGGGGGCAGAGGCCGGCTCGG
TGGTGGATGAGCGCGGCTTCGTATGGGAGAAGGCGGTCGAGGGCGACTTTTTCCGCGTGCAGTACAGC
GAAAGCAACCACTTGCACGTGGACCTGTGGCCCTTCTACCCCCGCAATGGCGTCATGACCAAGGACAC
GTGGCTGGACCACCGGCAGGATGTGGAGTTTCCCGAGCACTTCCTGCAGCCGCTGGTGCCCCTGCCCT
TTGCCGGCTTCGTGGCGCAGGCGCCTAACAACTACCGCCGCTTCCTGGAGCTCAAGTTCGGGCCCGGG
GTCATCGAGAACCCCCAGTACCCCAACCCGGCACTGCTGAGTCTGACGGGAAGCGGCTGA
FKRP amino acid sequence (495 aa)
NP_077277.1
SEQ ID NO: 3
MRLTRCQAALAAAITLNLLVLFYVSWLQHQPRNSRARGPRRASAAGPRVTVLVREFEAFDNAVPELVDSF
LQQDPAQPVVVAADTLPYPPLALPRIPNVRLALLQPALDRPAAASRPETYVATEFVALVPDGARAEAPGL
LERMVEALRAGSARLVAAPVATANPARCLALNVSLREWTARYGAAPAAPRCDALDGDAVVLLRARDLENL
SAPLARPVGTSLFLQTALRGWAVQLLDLTFAAARQPPLATAHARWKAEREGRARRAALLRALGIRLVSWE
GGRLEWFGCNKETTRCFGTVVGDTPAYLYEERWTPPCCLRALRETARYVVGVLEAAGVRYWLEGGSLLGA
ARHGDIIPWDYDVDLGIYLEDVGNCEQLRGAEAGSVVDERGFVWEKAVEGDFFRVQYSESNHLHVDLWPF
YPRNGVMTKDTWLDHRQDVEFPEHFLQPLVPLPFAGFVAQAPNNYRRFLELKEGPGVIENPQYPNPALLS
LTGSG
FST 344 DNA
SEQ ID NO: 4
atggtccgcgcgaggcaccagccggggggctttgcctcctgctgctgctgctctgccag
ttcatggaggaccgcagtgcccaggctgggaactgctggctccgtcaagcgaagaacggc
cgctgccaggtcctgtacaagaccgaactgagcaaggaggagtgctgcagcaccggccgg
ctgagcacctcgtggaccgaggaggacgtgaatgacaacacactcttcaagtggatgatt
ttcaacgggggcgcccccaactgcatcccctgtaaagaaacgtgtgagaacgtggactgt
ggacctgggaaaaaatgccgaatgaacaagaagaacaaaccccgctgcgtctgcgccccg
gattgttccaacatcacctggaagggtccagtctgcgggctggatgggaaaacctaccgc
aatgaatgtgcactcctaaaggcaagatgtaaagagcagccagaactggaagtccagtac
caaggcagatgtaaaaagacttgtcgggatgttttctgtccaggcagctccacatgtgtg
gtggaccagaccaataatgcctactgtgtgacctgtaatcggatttgcccagagcctgct
tcctctgagcaatatctctgtgggaatgatggagtcacctactccagtgcctgccacctg
agaaaggctacctgcctgctgggcagatctattggattagcctatgagggaaagtgtatc
aaagcaaagtcctgtgaagatatccagtgcactggtgggaaaaaatgtttatgggatttc
aaggttgggagaggccggtgttccctctgtgatgagctgtgccctgacagtaagtcggat
gagcctgtctgtgccagtgacaatgccacttatgccagcgagtgtgccatgaaggaagct
gcctgctcctcaggtgtgctactggaagtaaagcactccggatcttgcaactccatttcg
gaagacaccgaggaagaggaggaagatgaagaccaggactacagctttcctatatcttct
attctagagtgg
FST344 protein
SEQ ID NO: 5
MVRARHQPGGLCLLLL
LLCQFMEDRSAQAGNC
WLRQAKNGRCQVLYKT
ELSKEECCSTGRLSTS
WTEEDVNDNTLFKWMI
FNGGAPNCIPCKETCE
NVDCGPGKKCRMNKKN
KPRCVCAPDCSNITWK
GPVCGLDGKTYRNECA
LLKARCKEQPELEVQY
QGRCKKTCRDVFCPGS
STCVVDQTNNAYCVTC
NRICPEPASSEQYLCG
NDGVTYSSACHLRKAT
CLLGRSIGLAYEGKCI
KAKSCEDIQCTGGKKC
LWDFKVGRGRCSLCDE
LCPDSKSDEPVCASDN
ATYASECAMKEAACSS
GVLLEVKHSGSCNSIS
EDTEEEEEDEDQDYSF
PISSILEW
MHCK7 promoter
SEQ ID NO: 6
AGCTTGCATGTCTAAGCTAGACCCTTCAGATTAAAAATAACTGAGGTAAGGGCCTGGGTAGGGGAGGT
GGTGTGAGACGCTCCTGTCTCTCCTCTATCTGCCCATCGGCCCTTTGGGGAGGAGGAATGTGCCCAAG
GACTAAAAAAAGGCCATGGAGCCAGAGGGGCGAGGGCAACAGACCTTTCATGGGCAAACCTTGGGGCC
CTGCTGTCTAGCATGCCCCACTACGGGTCTAGGCTGCCCATGTAAGGAGGCAAGGCCTGGGGACACCC
GAGATGCCTGGTTATAATTAACCCAGACATGTGGCTGCCCCCCCCCCCCCAACACCTGCTGCCTCTAA
AAATAACCCTGTCCCTGGTGGATCCCCTGCATGCGAAGATCTTCGAACAAGGCTGTGGGGGACTGAGG
GCAGGCTGTAACAGGCTTGGGGGCCAGGGCTTATACGTGCCTGGGACTCCCAAAGTATTACTGTTCCA
TGTTCCCGGCGAAGGGCCAGCTGTCCCCCGCCAGCTAGACTCAGCACTTAGTTTAGGAACCAGTGAGC
AAGTCAGCCCTTGGGGCAGCCCATACAAGGCCATGGGGCTGGGCAAGCTGCACGCCTGGGTCCGGGGT
GGGCACGGTGCCCGGGCAACGAGCTGAAAGCTCATCTGCTCTCAGGGGCCCCTCCCTGGGGACAGCCC
CTCCTGGCTAGTCACACCCTGTAGGCTCCTCTATATAACCCAGGGGCACAGGGGCTGCCCTCATTCTA
CCACCACCTCCACAGCACAGACAGACACTCAGGAGCAGCCAGC
CMV promoter
SEQ ID NO: 7
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCAATGGGCGTGGA
TAGCGGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGTAGGC
GTGTACGGTGGGAGGTCTATATAAGCAGAGCTCGTTTAGTGAACCGTCAGA

### REFERENCES

1. High, K A, and Anguela, X M (2016). Adeno-associated viral vectors for the treatment of hemophilia. Hum Mol Genet 25: R36-41.
2. Samelson-Jones, B J, and Arruda, V R (2020). Translational Potential of Immune Tolerance Induction by AAV Liver-Directed Factor VIII Gene Therapy for Hemophilia A. Front Immunol 11: 618.
3. Michalakis, S, Gerhardt, M, Rudolph, G, Priglinger, S, and Priglinger, C (2021). Gene Therapy for Inherited Retinal Disorders: Update on Clinical Trials. Klin Monbl Augenheilkd 238: 272-281.
4. Testa, F, Maguire, A M, Rossi, S, Pierce, E A, Melillo, P, Marshall, K, et al. (2013). Three-year follow-up after unilateral subretinal delivery of adeno-associated virus in patients with Leber congenital Amaurosis type 2. Ophthalmology 120: 1283¬1291.
5. Mendell, J R, Sahenk, Z, Lehman, K, Nease, C, Lowes, L P, Miller, N F, et al. (2020). Assessment of Systemic Delivery of rAAVrh74.MHCK7.micro-dystrophin in Children With Duchenne Muscular Dystrophy: A Nonrandomized Controlled Trial JAMA Neurol.
6. Mendell, J R, Al-Zaidy, S, Shell, R, Arnold, W D, Rodino-Klapac, L R, Prior, T W, et al. (2017). Single-Dose Gene-Replacement Therapy for Spinal Muscular Atrophy. N Engl J Med 377: 1713-1722.
7. Brockington, M, Yuva, Y, Prandini, P, Brown, S C, Torelli, S, Benson, M A, et al. (2001). Mutations in the fukutin-related protein gene (FKRP) identify limb girdle muscular dystrophy 21 as a milder allelic variant of congenital muscular dystrophy MDC1C.
8. Chan, Y M, Keramaris-Vrantsis, E, Lidov, H G, Norton, J H, Zinchenko, N, Gruber, H E, et al. (2010). Fukutin-related protein is essential for mouse muscle, brain and eye development and mutation recapitulates the wide clinical spectrums of dystroglycanopathies. Hum Mol Genet 19: 3995-4006.
9. Brockington, M, Blake, D J, Prandini, P, Brown, S C, Torelli, S, Benson, M A, et al. (2001). Mutations in the fukutin-related protein gene (FKRP) cause a form of congenital muscular dystrophy with secondary laminin alpha2 deficiency and abnormal glycosylation of alpha-dystroglycan. Am J Hum Genet 69: 1198-1209.
10. Brown, S C, Torelli, S, Brockington, M, Yuva, Y, Jimenez, C, Feng, L, et al. (2004). Abnormalities in alpha-dystroglycan expression in MDC1C and LGMD21 muscular dystrophies. Am J Pathol 164: 727-737.
11. Ervasti, J M, and Campbell, K P (1991). Membrane organization of the dystrophin-glycoprotein complex. Cell 66: 1121-1131.
12. Akhavan, A, Crivelli, S N, Singh, M, Lingappa, V R, and Muschler, J L (2008). SEA domain proteolysis determines the functional composition of dystroglycan. FASEB J 22: 612-621.
13. Ervasti, J M, and Campbell, K P (1993). A role for the dystrophin-glycoprotein complex as a transmembrane linker between laminin and actin. J Cell Biol 122: 809-823.
14. Michele, D E, and Campbell, K P (2003). Dystrophin-glycoprotein complex: post-translational processing and dystroglycan function. J Biol Chem 278: 15457-15460.
15. Michele, D E, Barresi, R, Kanagawa, M, Saito, F, Cohn, R D, Satz, J S, et al. (2002). Post-translational disruption of dystroglycan-ligand interactions in congenital muscular dystrophies. Nature 418: 417-422.
16. Yoshida-Moriguchi, T, and Campbell, K P (2015). Matriglycan: a novel polysaccharide that links dystroglycan to the basement membrane. Glycobiology 25: 702-713.
17. Inamori, K, Yoshida-Moriguchi, T, Hara, Y, Anderson, M E, Yu, L, and Campbell, K P (2012). Dystroglycan function requires xylosyl- and glucuronyltransferase activities of LARGE. Science 335: 93-96.
18. Nilsson, J, Nilsson, J, Larson, G, and Grahn, A (2010). Characterization of site-specific O-glycan structures within the mucin-like domain of alpha-dystroglycan from human skeletal muscle. Glycobiology 20: 1160-1169.
19. Hara, Y, Kanagawa, M, Kunz, S, Yoshida-Moriguchi, T, Satz, J S, Kobayashi, Y M, et al. (2011). Like-acetylglucosaminyltransferase (LARGE)-dependent modification of dystroglycan at Thr-317/319 is required for laminin binding and arenavirus infection. Proc Natl Acad Sci USA 108: 17426-17431.
20. Kanagawa, M, Kobayashi, K, Tajiri, M, Manya, H, Kuga, A, Yamaguchi, Y, et al. (2016). Identification of a Post-translational Modification with Ribitol-Phosphate and Its Defect in Muscular Dystrophy. Cell Rep 14: 2209-2223.
21. Xu, L, Lu, P J, Wang, C H, Keramaris, E, Qiao, C, Xiao, B, et al. (2013). Adeno-associated virus 9 mediated FKRP gene therapy restores functional glycosylation of alpha-dystroglycan and improves muscle functions. Mol Ther 21: 1832-1840.
22. Qiao, C, Wang, C H, Zhao, C, Lu, P, Awano, H, Xiao, B, et al. (2014). Muscle and heart function restoration in a limb girdle muscular dystrophy 21 (LGMD2I) mouse model by systemic FKRP gene delivery. Mol Ther 22: 1890-1899.
23. Awano, H, Blaeser, A, Keramaris, E, Xu, L, Tucker, J, Wu, B, et al. (2015). Restoration of Functional Glycosylation of alpha-Dystroglycan in FKRP Mutant Mice Is Associated with Muscle Regeneration. Am J Pathol 185: 2025-2037.
24. Gicquel, E, Maizonnier, N, Foltz, S J, Martin, W J, Bourg, N, Svinartchouk, F, et al. (2017). AAV-mediated transfer of FKRP shows therapeutic efficacy in a murine model but requires control of gene expression. Hum Mol Genet 26: 1952-1965.
25. Kanagawa, M, and Toda, T (2017). Muscular Dystrophy with Ribitol-Phosphate Deficiency: A Novel Post-Translational Mechanism in Dystroglycanopathy. J Neuromuscul Dis 4: 259-267.
26. Wu, B, Drains, M, Shah, S N, Lu, P J, Leroy, V, Killilee, J, et al. (2022). Ribitol dose-dependently enhances matriglycan expression and improves muscle function with prolonged life span in limb girdle muscular dystrophy 21 mouse model. PLoS One 17: e0278482.
27. Bailey, E C, Alrowaished, S S, Kilroy, E A, Crooks, E S, Drinkert, D M, Karunasiri, C M, et al. (2019). NAD+improves neuromuscular development in a zebrafish model of FKRP-associated dystroglycanopathy. Skelet Muscle 9: 21.
28. Cataldi, M P, Blaeser, A, Lu, P, Leroy, V, and Lu, Q L (2020). ISPD Overexpression Enhances Ribitol-Induced Glycosylation of alpha-Dystroglycan in Dystrophic FKRP Mutant Mice. Mol Ther Methods Clin Dev 17: 271-280.
29. Vannoy, C H, Xiao, W, Lu, P, Xiao, X, and Lu, Q L (2017). Efficacy of Gene Therapy Is Dependent on Disease Progression in Dystrophic Mice with Mutations in the FKRP Gene. Mol Ther Methods Clin Dev 5: 31-42.
30. Vannoy, C H, Leroy, V, and Lu, Q L (2018). Dose-Dependent Effects of FKRP Gene-Replacement Therapy on Functional Rescue and Longevity in Dystrophic Mice. Mol Ther Methods Clin Dev 11: 106-120.
31. Amthor, H, Nicholas, G, McKinnell, I, Kemp, C F, Sharma, M, Kambadur, R, et al. (2004). Follistatin complexes Myostatin and antagonises Myostatin-mediated inhibition of myogenesis. Dev Biol 270: 19-30.
32. McPherron, A C, and Lee, S J (1997). Double muscling in cattle due to mutations in the myostatin gene. Proc Natl Acad Sci USA 94: 12457-12461.
33. McPherron, A C, Lawler, A M, and Lee, S J (1997). Regulation of skeletal muscle mass in mice by a new TGF-beta superfamily member. Nature 387: 83-90.
34. Wagner, K R, McPherron, A C, Winik, N, and Lee, S J (2002). Loss of myostatin attenuates severity of muscular dystrophy in mdx mice. Ann Neurol 52: 832-836.
35. Bogdanovich, S, Krag, T O, Barton, E R, Morris, L D, Whittemore, L A, Ahima, R S, et al. (2002). Functional improvement of dystrophic muscle by myostatin blockade. Nature 420: 418-421.
36. Schuelke, M, Wagner, K R, Stolz, L E, Hubner, C, Riebel, T, Komen, W, et al. (2004). Myostatin mutation associated with gross muscle hypertrophy in a child. N Engl J Med 350: 2682-2688.
37. Cohn, R D, Liang, H Y, Shetty, R, Abraham, T, and Wagner, K R (2007). Myostatin does not regulate cardiac hypertrophy or fibrosis. Neuromuscul Disord 17: 290-296.
38. Amthor, H, Macharia, R, Navarrete, R, Schuelke, M, Brown, S C, Otto, A, et al. (2007). Lack of myostatin results in excessive muscle growth but impaired force generation. Proc Natl Acad Sci USA 104: 1835-1840.
39. Dumonceaux, J, Marie, S, Beley, C, Trollet, C, Vignaud, A, Ferry, A, et al. (2010). Combination of myostatin pathway interference and dystrophin rescue enhances tetanic and specific force in dystrophic mdx mice. Mol Ther 18: 881-887.
40. Leung, D G, Bocchieri, A E, Ahlawat, S, Jacobs, M A, Parekh, V S, Braverman, V, et al. (2021). A phase Ib/IIa, open-label, multiple ascending-dose trial of domagrozumab in fukutin-related protein limb-girdle muscular dystrophy. Muscle Nerve 64: 172-179.
41. Lee, S J (2007). Quadrupling muscle mass in mice by targeting TGF-beta signaling pathways. PLoS One 2: e789.
42. Winbanks, C E, Weeks, K L, Thomson, R E, Sepulveda, P V, Beyer, C, Qian, H, et al. (2012). Follistatin-mediated skeletal muscle hypertrophy is regulated by Smad3 and mTOR independently of myostatin. J Cell Biol 197: 997-1008.
43. Bodine, S C, Stitt, T N, Gonzalez, M, Kline, W O, Stover, G L, Bauerlein, R, et al. (2001). Akt/mTOR pathway is a crucial regulator of skeletal muscle hypertrophy and can prevent muscle atrophy in vivo. Nat Cell Biol 3: 1014-1019.
44. Mendell, J R, Sahenk, Z, Al-Zaidy, S, Rodino-Klapac, L R, Lowes, L P, Alfano, L N, et al. (2017). Follistatin Gene Therapy for Sporadic Inclusion Body Myositis Improves Functional Outcomes. Mol Ther 25: 870-879.
45. Mendell, J R, Sahenk, Z, Malik, V, Gomez, A M, Flanigan, K M, Lowes, L P, et al. (2015). A phase 1/2a follistatin gene therapy trial for becker muscular dystrophy. Mol Ther 23: 192-201.
46. Delluc-Clavieres, A, Le Bec, C, Van den Berghe, L, Conte, C, Allo, V, Danos, O, et al. (2008). Efficient gene transfer in skeletal muscle with AAV-derived bicistronic vector using the FGF-1 IRES. Gene Ther 15: 1090-1098.
47. Gray, S J, Foti, S B, Schwartz, J W, Bachaboina, L, Taylor-Blake, B, Coleman, J, et al. (2011). Optimizing promoters for recombinant adeno-associated virus-mediated gene expression in the peripheral and central nervous system using self-complementary vectors. Hum Gene Ther 22: 1143-1153.
48. Xu, R, Jia, Y, Zygmunt, D A, Cramer, M L, Crowe, K E, Shao, G, et al. (2018). An Isolated Limb Infusion Method Allows for Broad Distribution of rAAVrh74.MCK.GALGT2 to Leg Skeletal Muscles in the Rhesus Macaque. Mol Ther Methods Clin Dev 10: 89-104.
49. Salva, M Z, Himeda, C L, Tai, P W, Nishiuchi, E, Gregorevic, P, Allen, J M, et al. (2007). Design of tissue-specific regulatory cassettes for high-level rAAV-mediated expression in skeletal and cardiac muscle. Mol Ther 15: 320-329.
50. Rodino-Klapac, L R, Haidet, A M, Kota, J, Handy, C, Kaspar, B K, and Mendell, J R (2009). Inhibition of myostatin with emphasis on follistatin as a therapy for muscle disease. Muscle Nerve 39: 283-296.
51. Packer, D, and Martin, P T (2021). Micro-laminin gene therapy can function as an inhibitor of muscle disease in the dy(W) mouse model of MDC1A. Mol Ther Methods Clin Dev 21: 274-287.
52. Weinmann, J, Weis, S, Sippel, J, Tulalamba, W, Remes, A, El Andari, J, et al. (2020). Identification of a myotropic AAV by massively parallel in vivo evaluation of barcoded capsid variants. Nat Commun 11: 5432.
53. Goddeeris, M M, Wu, B, Venzke, D, Yoshida-Moriguchi, T, Saito, F, Matsumura, K, et al. (2013). LARGE glycans on dystroglycan function as a tunable matrix scaffold to prevent dystrophy. Nature 503: 136-140.
54. Cohn, R D, Henry, M D, Michele, D E, Barresi, R, Saito, F, Moore, S A, et al. (2002). Disruption of DAG1 in differentiated skeletal muscle reveals a role for dystroglycan in muscle regeneration. Cell 110: 639-648.
55. Krag, T O, Hauerslev, S, Sveen, M L, Schwartz, M, and Vissing, J (2011). Level of muscle regeneration in limb-girdle muscular dystrophy type 21 relates to genotype and clinical severity. Skelet Muscle 1: 31.
56. Nakatani, M, Takehara, Y, Sugino, H, Matsumoto, M, Hashimoto, O, Hasegawa, Y, et al. (2008). Transgenic expression of a myostatin inhibitor derived from follistatin increases skeletal muscle mass and ameliorates dystrophic pathology in mdx mice. FASEB J 22: 477-487.
57. Chandraskeharan, K, and Martin, P T (2009). Embryonic overexpression of Galgt2 inhibits skeletal muscle growth via activation of myostatin signaling. Muscle Nerve 39: 25-41.
58. Rodino-Klapac, L R, Janssen, P M, Shontz, K M, Canan, B, Montgomery, C L, Griffin, D, et al. (2013). Micro-dystrophin and follistatin co-delivery restores muscle function in aged DMD model. Hum Mol Genet 22: 4929-4937.
59. Abmayr, S, Gregorevic, P, Allen, J M, and Chamberlain, J S (2005). Phenotypic improvement of dystrophic muscles by rAAV/microdystrophin vectors is augmented by Igf1 codelivery. Mol Ther 12: 441-450.
60. Willis, T A, Hollingsworth, K G, Coombs, A, Sveen, M L, Andersen, S, Stojkovic, T, et al. (2013). Quantitative muscle MRI as an assessment tool for monitoring disease progression in LGMD21: a multicentre longitudinal study. PLoS One 8: e70993.
61. Xiao, X, Li, J, and Samulski, R J (1998). Production of high-titer recombinant adeno-associated virus vectors in the absence of helper adenovirus. J Virol 72: 2224-2232.
62. Clark, K R, Liu, X, McGrath, J P, and Johnson, P R (1999). Highly purified recombinant adeno-associated virus vectors are biologically active and free of detectable helper and wild-type viruses. Human gene therapy 10: 1031-1039.
63. Flanigan, K M, Vetter, T A, Simmons, T R, lammarino, M, Frair, E C, Rinaldi, F, et al. (2022). A first-in-human phase I/IIa gene transfer clinical trial for Duchenne muscular dystrophy using rAAVrh74.MCK.GALGT2. Mol Ther Methods Clin Dev 27: 47-60.
64. Vetter, T A, Nicolau, S, Bradley, A J, Frair, E C, and Flanigan, K M (2022). Automated immunofluorescence analysis for sensitive and precise dystrophin quantification in muscle biopsies. Neuropathol Appl Neurobiol 48: e12785.
65. Livak, K J, and Schmittgen, T D (2001). Analysis of relative gene expression data using real-time quantitative PCR and the 2(-Delta Delta C(T)) Method. Methods 25: 402-408.

1. A polynucleotide sequence comprising in 5′ to 3′ order: an AAV ITR, a first transcriptional control sequence, an enhancer and/or intron, a first transgene sequence, a polyadenylation signal sequence, a second transcriptional control sequence, a second transgene sequence, a polyadenylation signal sequence and an AAV ITR.

2. The polynucleotide sequence of claim 1 wherein the first transcriptional control sequence is a muscle specific promoter.

3. The polynucleotide sequence of claim 2, wherein the muscle-specific promoter comprises one or more of a human skeletal actin gene element, a cardiac actin gene element, a desmin promoter, a skeletal alpha-actin (ASKA) promoter, a troponin I (TNN12) promoter, a myocyte-specific enhancer binding factor MEF binding element, a muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, a murine creatine kinase enhancer element, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE).

4. The polynucleotide sequence of any one of claims 1-3, wherein the first transgene sequence encodes a protein that is reduced or eliminated in a subject suffering from a degenerative muscular disorder.

5. The polynucleotide sequence of any one of claims 1-4, wherein the second transgene sequence encodes a muscle building protein.

6. A polynucleotide sequence comprising in 5′ to 3′ order: an AAV ITR, a first transcriptional control sequence operably linked to a nucleotide sequence encoding the fukutin-related protein (FKRP) and a second transcriptional control sequence operably linked to a nucleotide sequence encoding FST (follistatin 344) and an AAV ITR.

7. The polynucleotide sequence of claim 6, wherein the nucleotide sequence encoding the FKRP protein comprises the nucleotides of 2056-2846 of SEQ ID NO: 1.

8. The polynucleotide sequence of claim 6 or 7, where the nucleotide sequence encoding FST (follistatin 344) comprises nucleotides 5393-6427 of SEQ ID NO: 1.

9. The polynucleotide sequence of any one of claims 6-8, wherein the first transcriptional control sequence is the MHCK7 promoter.

10. The polynucleotide sequence of any one of claims 6-9, wherein the second transcriptional control sequence is the CMV promoter and/or the CMV enhancer.

11. The polynucleotide sequence of any one of claims 6-10, further comprising a SV40 enhancer and/or an intron.

12. A polynucleotide sequence comprising a nucleotide sequence that is at least 95% identical to nucleotides 1847 to 6672 of SEQ ID NO: 1.

13. The polynucleotide sequence of claim 12 wherein the nucleotide sequence comprises nucleotides 1847 to 6672 of SEQ ID NO: 1.

14. A recombinant adeno-associated virus (rAAV) comprising the polynucleotide sequence of any one of claims 1-13.

15. The rAAV of any one of claim 14, wherein the rAAV comprises AAV-1, AAV-2, AAV-3, AAV-4, AAV-5, AAV-6, AAV-7, AAV-8, AAV-9, AAV-10, AAV-11, AAV-12, AAV-13, AAVrh.74, AAVrh.10, Anc80, AAV7m8, AAVMyo, MYOAAV capsid protein, or a variant thereof.

16. A recombinant AAV particle comprising the polynucleotide sequence of any one of claims 1-13 or the rAAV of claim 14 or 15.

17. A composition comprising the rAAV of claim 14 or 15 or the rAAV particle of claim 16.

18. A method of treating muscular dystrophy comprising administering a rAAV of claim 14 or 15, the rAAV particle of claim 16 or the composition of claim 17 to a subject in need thereof.

19. The method of claim 18, wherein the muscular dystrophy is limb girdle muscular dystrophy 2I or congenital muscular dystrophy 1C.

20. The method of claim 17 or 18, wherein the rAAV, rAAV particle or composition is administered using systemic administration, intramuscular injection or intravenous injection.

21. A composition for treating muscular dystrophy in a subject in need thereof, wherein the composition comprises rAAV of claim 14 or 15, the rAAV particle of claim 16 or the composition of claim 17.

22. The composition of claim 21, wherein the muscular dystrophy is limb girdle muscular dystrophy 2I or congenital muscular dystrophy 1C.

23. The composition of claim 21 or 22, wherein the composition is formulated for systemic administration, intramuscular injection or intravenous injection.

24. Use of a rAAV of claim 14 or 15, the rAAV particle of claim 16 or the composition of claim 17 for the preparation of a medicament for treating muscular dystrophy.

25. The use of claim 24, wherein the muscular dystrophy is limb girdle muscular dystrophy 2I or conexamplegenital muscular dystrophy 1C.

26. The use of claim 24 or 25, wherein the medicament is formulated for systemic administration, intramuscular injection or intravenous injection.


SEQUENCE LISTING (7 sequences)

SEQ ID NO: 1 - DNA, 8518 bp, synthetic construct
  source 1..8518: mol_type = other DNA
  misc_difference 83..899: note = KanR
  misc_difference 1021..1609: note = Ori
  misc_difference 1847..2013: note = Wild type AAV ITR
  misc_difference 2056..2846: note = MHCK7 promoter
  misc_difference 2858..3004: note = Partial SV40 enhancer/intron
  misc_difference 3016..3026: note = Kozak
  misc_difference 3024..4511: note = Human FKRP cDNA
  misc_difference 4532..4580: note = Poly A
  misc_difference 4591..5117: note = CMV promoter/enhancer
  misc_difference 5188..5382: note = SV40 enhancer with intron
  misc_difference 5393..6426: note = FST344 cDNA
  misc_difference 6442..6491: note = Poly A
  misc_difference 6506..6672: note = Wild type AAV ITR
  misc_difference 6675..6743: note = LacZ alpha
  misc_difference 7061..7367: note = F1 ori
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgagcatgcactagtgcggccgcaataaaagatctttattttcattagatctgtgtgttggttttttgtgtgtctagagcttcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaaactagtgcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 2 - DNA, 1488 bp, synthetic construct
  source 1..1488: mol_type = other DNA
atgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctga

SEQ ID NO: 3 - protein, 495 aa, synthetic construct
MRLTRCQAALAAAITLNLLVLFYVSWLQHQPRNSRARGPRRASAAGPRVTVLVREFEAFDNAVPELVDSFLQQDPAQPVVVAADTLPYPPLALPRIPNVRLALLQPALDRPAAASRPETYVATEFVALVPDGARAEAPGLLERMVEALRAGSARLVAAPVATANPARCLALNVSLREWTARYGAAPAAPRCDALDGDAVVLLRARDLFNLSAPLARPVGTSLFLQTALRGWAVQLLDLTFAAARQPPLATAHARWKAEREGRARRAALLRALGIRLVSWEGGRLEWFGCNKETTRCFGTVVGDTPAYLYEERWTPPCCLRALRETARYVVGVLEAAGVRYWLEGGSLLGAARHGDIIPWDYDVDLGIYLEDVGNCEQLRGAEAGSVVDERGFVWEKAVEGDFFRVQYSESNHLHVDLWPFYPRNGVMTKDTWLDHRQDVEFPEHFLQPLVPLPFAGFVAQAPNNYRRFLELKFGPGVIENPQYPNPALLSLTGSG

SEQ ID NO: 4 - DNA, 1032 bp, synthetic construct
  source 1..1032: mol_type = other DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtgg

SEQ ID NO: 5 - protein, 344 aa, synthetic construct
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 6 - DNA, 791 bp, synthetic construct
  source 1..791: mol_type = other DNA
agcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 7 - DNA, 527 bp, synthetic construct
  source 1..527: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaga